(19) United States
(12) Patent Application Publication
(10) Pub. No.: US 2026/0001933 A1
(43) Pub. Date: Jan. 1, 2026

(54) PROTEINS COMPRISING THE EXTRACELLULAR DOMAIN OF P75NTR

(71) Applicant: PETMEDIX LTD., Cambridge (GB)

(72) Inventors: Meng Amy LI, Cambridge (GB); Marco BARDELLI, Cambridge (GB)

(21) Appl. No.: 18/875,406

(22) PCT Filed: Oct. 21, 2022
(86) PCT No.: PCT/GB2022/005269

§ 371 Date: Dec. 16, 2024

(30) Foreign Application Priority Data
Oct. 21, 2021 (GB) 2115098.2

(51) Int. Cl. C07K 14/705; A61K 38/00; A61K 45/06; A61P 25/04

(52) U.S. Cl. CPC C07K 14/70571; A61K 45/06; A61P 25/04; A61K 38/00; C07K 2319/30

(57) ABSTRACT

The present invention relates to effective pain therapies in companion animals. In particular, an isolated companion animal p75NTR protein or a fusion protein containing the same or portions thereof are contemplated. Nucleic acids encoding the proteins are also encompassed in the invention as well as methods of using the same.

### RELATED APPLICATIONS AND INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to effective pain therapies in companion animals.

### INTRODUCTION

There is a huge need for therapies in veterinary medicine, in particular for pain relief.

Pain relief treatments for dogs currently include nonsteroidal anti-inflammatory drugs, commonly called NSAIDs, and several nonsteroidal anti-inflammatory drugs which help to control pain and inflammation associated with osteoarthritis. Several NSAIDs have been approved by the FDA. However, there is a need for further effective pain treatments in companion animals with minimal side effects.

Nerve growth factor (NGF) was firstly discovered in 1950s. The NGF protein was then cloned and identified as part of brain-derived neurotrophic factor (BDNF), neurotrophin-3 and 4/5 (NT-3, NT-4-5). All of these are secreted proteins that promote survival and growth of the peripheral nervous system.

NGF causes peripheral sensitization both in vitro and in vivo, as illustrated by the increased response of DRG neurons to temperature or capsaicin in its presence. NGF also leads to transcriptional regulation after retrograde axonal transport, as illustrated by immunostaining showing upregulation of BDNF after intrathecal NGF treatment. Furthermore, NGF can cause sprouting of peripheral afferents into diseased joints and cancerous tissue (Denk et al, Annual Review of Neuroscience, Vol. 40:307-325, 2017).

NGF is expressed at low levels in adulthood, but injury, inflammation or release of NGF cause activation of inflammatory cells. These cells in turn produce and secrete NGF as well and this leads to short-term and long-term effects. NGF has a well-known and multifunctional role in nociceptive processing, although the precise signaling pathways downstream of NGF receptor activation that mediate nociception are complex and not completely understood. The role of NGF in nociception and the generation and/or maintenance of chronic pain has led to it becoming an attractive target of pain therapeutics for the treatment of chronic pain conditions (Barker et al, Journal of Pain Research, 2020:13 1223-1241).

Very low doses of monoclonal antibodies (mAbs) directed against NGF can reduce chronic pain. However, during clinical trials in humans, a small subset of patients treated with mAbs directed against NGF developed rapidly progressive joint degeneration due to interaction with NSAIDs treatment. Complete NGF removal is shown to cause impaired bone and cartilage repairing (Denk et al, Annual Review of Neuroscience, Vol. 40:307-325, 2017). Treatments for use in dogs and cats based on species-specific mAbs that target NGF are now being developed for the management of osteoarthritis (OA)-associated pain (Enomoto et al, Vet Rec. 2019 Jan. 5; 184(1):23 and WO2019177690). However, given the side effects that occurred in clinical trials in humans, there is a need to develop alternative treatments that target NGF.

Citation or identification of any document in this application is not an admission that such document is available as prior art to the present invention.

### SUMMARY OF THE INVENTION

The invention is aimed at addressing a need for effective pain therapies in companion animals.

The invention provides a pain therapy for companion animals with reduced side effects compared to anti-NGF antibody therapies. The proteins of the invention bind NGF which is at elevated levels in pain conditions, thus binding to excess NGF to restore normal NGF levels without completely blocking NGF signalling. Without wishing to be bound by theory, the inventors believe that this ensures a level of NGF signalling which is required for healthy functions. Furthermore, it is believed that the fusion proteins of the invention can operate at a very low dose, but are highly efficacious.

The inventors have used an analgesic strategy to reduce, but not completely deplete, NGF in circulation. To this end, the Extracellular Domain (ECD) of p75 neurotrophin receptor (p75NTR) was used, fused to Fc to increase its half-life. p75NTR binds NGF and other brain-derived neurotrophic factors (NT3, NT4) and mediates different cellular activities.

The use of human p75 has been described for treatment of pain in humans (WO2013136078).

Thus, in a first aspect, the invention relates to an isolated companion animal p75NTR protein or a portion thereof. The invention also relates to an isolated nucleic acid encoding the protein or portion thereof.

The invention also relates to a vector comprising a nucleic acid as described above.

The invention further relates to a host cell comprising a nucleic acid as described above.

In another aspect, the invention relates to a fusion protein comprising an isolated companion animal p75NTR extracellular domain or portion thereof and a half-life extending moiety.

In another aspect, the invention relates to a nucleic acid encoding a fusion protein as described above.

In another aspect, the invention relates to a vector comprising a nucleic acid as described above.

In another aspect, the invention relates to a host cell comprising a nucleic acid as described above or a vector as described above.

In another aspect, the invention relates to a pharmaceutical composition comprising an isolated companion animal p75NTR protein as described above and herein, or a fusion protein as described above and herein.

In another aspect, the invention relates to a method for treating an NGF-related disorder in a companion animal comprising administering an isolated companion animal p75NTR protein as described above, a fusion protein as described above and herein or a pharmaceutical composition as described above and herein.

In another aspect, the invention relates to the use of an isolated companion animal p75NTR protein as described above, a fusion protein as described above or a pharmaceutical composition as described above and herein in the treatment of an NGF-related disorder in a companion animal.

In another aspect, the invention relates to a method of inhibiting NGF activity in a companion animal comprising administering an isolated companion animal p75NTR protein as described above, a fusion protein as described above and herein or a pharmaceutical composition as described above and herein.

In another aspect, the invention relates to a kit comprising an isolated companion animal p75NTR protein as described above, a fusion protein as described above or a pharmaceutical composition as described above and optionally instructions for use.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DESCRIPTION OF FIGURES

The invention is described in the following non-limiting figures.

FIG. 1. Exemplary fusion proteins (a) PetML119 and (b) PetML122.

FIG. 2. a) Sequence alignment of part of the ECD of p75NTR as used in the fusion proteins of the invention. There is a very high similarity between species:
Canine vs Feline: 1/164 (more than 99% identical);
Canine vs Equine: 4/164 (97.6% identical);
Feline vs Equine: 5/164 (97% identical);
Canine vs Bovine: 2/164 (98.8% identical)
b) Sequence alignment of the Fc region (including CH3, CH2 and CH2-CH1 hinge) from different species/different isotypes.

FIG. 3. Purity of isolated fusion proteins. a) H-SEC b) H-SCX. PetML119 appears to be highly pure by analytical size exclusion chromatography. b) Cation exchange chromatography showed presence of one main species (˜80%) and two minor charge variants.

FIG. 4. PetML119 Stability.
a) Tonset analysis.
b) Thermal and chemical stress.
c) Freeze and thaw stress.
d) Thermal stability.
e) Aggregation analysis.

FIG. 5. Protein A binding.
a) and b) PetML119 Protein A binding.
c) and d) PetML122 Protein A binding.
e) Bedinvetmab Protein A binding. Bedinvetmab is an anti-NGF IgG control.
f) Table showing kinetics.

FIG. 6. PetML119/122 canine and murine FcRN binding.
a) Canine FcRN binding PetML119.
b) Murine FcRN binding PetML119.
c) Affinity table of canine and murine binding FcRN.
d) Canine FcRN binding PetML122.
e) Murine FcRN binding PetML122.
f) Affinity table of canine and murine FcRN binding.

FIG. 7. NGF binding.
a) PetML119 binding to human NGF.
b) PetML119 to rat NGF.
c) Kinetics affinity table.
d) Bedinvetmab binding to human NGF.
e) Kinetics affinity table.

FIG. 8. TF-1 cell proliferation assay. TF-1 labelled with CFSE-cell trace at 2.5 uM 30′ RT. TF-1 seeded at 104 cells/well in 96 well plate. Positive ctr: TF-1 cells in RPMI media+10% FBS+10 ng/mL hNGF 3 timepoints (t 2 hrs, t 4 d and t 7 d).
a) Activity of NGF: 2 ng/mL EC50.
b) TF-1 cell proliferation assay read out.
c) TF-1 cell proliferation assay read out using PetML119, PetML122 and Bedinvetmab.

FIG. 9. In vivo model.

FIG. 10. Analgesic effects in vivo.
a) 2 hours post IV treatment.
b) 3 days post IV treatment.
c) 11 days post IV treatment.
d) 18 days post IV treatment.

FIG. 11. Joint Diametre and body weight.
a) Joint Diametre analysis.
b) body weight analysis.

FIG. 12. pk analysis—half life.
a) Standard curve for PetML119 in rat serum.
b) Standard curve for PetML122 in rat serum.
c-f) Rat study pK results. Calculated half-life for PetML119 at different concentration is between 56-70 hrs.

FIG. 13. Safety analysis of PetML119/PetML122 in dogs.
a) Body weight. Top row in top table shows the individual animals in Group 1, bottom row in top table shows the individual animals in Group 2. Animals in Group 1 were treated with PetML119, animals in Group 2 were treated with PetML122 (see Table 3). Bottom table shows mean values.
b) Anti-Drug Antibodies (ADA) analysis. Top panel PetML119, bottom panel PetML120.

FIG. 14. a-d Efficacy analysis of PetML119/PetML122 in dogs.

Force plate analysis and visual lameness analysis of PetML119/PetML122.

FIG. 15. pk analysis—half life for dog.

Top (a) shows endpoint Absorbance at 650 nm and 450 nm using sera. Bottom (b) shows one phase decay fitting to estimate half-life for PetML119/PetML122.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols. 1 and 2, Ontermann and Duebel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The invention provides biological therapeutics for veterinary use, in particular fusion proteins for use in the treatment of companion animals such as dogs, cats, bovines or horses.

In a first aspect, the invention relates to an isolated companion animal p75NTR protein or a portion thereof.

As used herein, the term p75NTR protein refers to a p75NTR protein that binds NGF and/or other neurotrophins (BDNF, NT-3 and/or NT-4/5). As used herein, this means that the protein is capable of binding to NGF and inhibiting NGF biological activity and/or downstream pathway(s) mediated by NGF signalling. An NGF binding protein reduces NGF biological activity, including downstream pathways mediated by NGF signalling and/or reduces the amount of NGF that is in circulation and which can bind to its receptors trkA and NGFR (p75NTR).

The term companion animal as used herein refers to a dog, cat or horse. In one embodiment, the companion animal is a dog. In another embodiment, the animal to be treated may be a cow or pig.

The term “isolated” protein or polypeptide refers to a protein or polypeptide that is substantially free of other proteins or polypeptides, having different antigenic specificities. Moreover, protein or polypeptide may be substantially free of other cellular material and/or chemicals. Thus, the protein, nucleic acids and polypeptides described herein are preferably isolated. Thus, as used herein, an “isolated” protein, or polypeptide means protein or polypeptide that has been identified and separated and/or recovered from a component of its natural cell culture environment. Contaminant components of its natural environment are materials that would interfere with diagnostic or therapeutic uses for the protein or polypeptide, and may include enzymes, hormones, and other proteinaceous or non-proteinaceous solutes.

The terms “polypeptide” and “protein” are used interchangeably to refer to a polymer of amino acid residues and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational and post-translational modifications of the polypeptide, such as, for example, disulfide-bond formation, glycosylation, acetylation, phosphorylation, proteolytic cleavage (e.g., cleavage by furins or metalloproteases and prohormone convertases (PCs)), and the like. Furthermore, for purposes of the present invention, a “polypeptide” encompasses a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, have a well-defined conformation.

Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

By “amino acid” herein is meant one of the 20 naturally occurring amino acids or any non-natural analogues that may be present at a specific, defined position. Amino acid encompasses both naturally occurring and synthetic amino acids. Although in most cases, when the protein is to be produced recombinantly, only naturally occurring amino acids are used.

In one embodiment, the isolated companion animal p75NTR protein comprises or consists of a canine, feline or bovine p75NTR protein or a portion or a variant thereof. Therefore, in one embodiment, the isolated companion animal p75NTR protein comprises or consists of SEQ ID No. 1, 3, 5 or 36, a portion or a variant thereof or a variant of a portion.

The p75 neurotrophin receptor p75NTR in its native form exists as a transmembrane glycoprotein. Family members are characterised by multiple cysteine-rich domains for ligand binding, a single transmembrane sequence extracellular domain (ECD), and a non-catalytic cytoplasmic domain.

Endogenous soluble ECD of p75NTR is produced by regulated proteolysis by α-secretase and γ-secretase that cleaves the protein near the membrane junction of the ECD. This is cleavage results in the release of the cytoplasmic domain which is free to bind NGF as a natural antagonist to NGF signalling.

In one embodiment, the isolated companion animal p75NTR protein or a portion thereof comprises or consists of the extracellular domain (ECD) or part thereof or a variant thereof. In one embodiment, the isolated companion animal p75NTR protein or a portion thereof comprises or consists of the extracellular domain (ECD). In one embodiment, the isolated companion animal p75NTR protein or a portion thereof comprises the extracellular domain (ECD) and additional C-terminal amino acids of the companion animal p75NTR protein adjacent to the ECD. For example, the portion of the companion animal p75NTR protein may comprise the ECD and at least 1-5 or 5-10 amino acids, e.g. 1, 2, 3, 4, 5, 6, 7, 8, 9 or 10 C-terminal amino acids of the companion animal p75NTR protein adjacent to the ECD; or the ECD and at least 10-20 amino acids, or the ECD and at least 20-30 amino acids, or the ECD and at least 30-40 amino acids, or the ECD and at least 40-50 amino acids, or the ECD and at least 50-60 amino acids, or the ECD and at least 60-70 amino acids, or the ECD and at least 70-80 amino acids, or the ECD and at least 80-90 amino acids, or the ECD and at least 90-100 amino acids, or the ECD and at least 100-110 amino acids, or the ECD and at least 110-120 amino acids, or the ECD and at least 120-130 amino acids, or the ECD and at least 130-140 amino acids, or the ECD and at least 140-150 amino acids, or the ECD and at least 150-160 amino acids, or the ECD and at least 160-170 amino acids or the ECD and at least 170-180 amino acids C-terminal amino acids of the companion animal p75NTR protein adjacent to the ECD.

In one embodiment, α-secretase and γ-secretase cleavage sites within the ECD are removed. In one embodiment, the stalk region is removed. In one embodiment, the stalk region and α-secretase and γ-secretase cleavage sites within the ECD are removed.

In one embodiment, the isolated companion animal p75NTR extracellular domain is canine and comprises or consists of SEQ ID No. 7 or a variant thereof or a portion thereof, for example SEQ ID No. 34.

In another embodiment, the isolated companion animal p75NTR extracellular domain is feline and comprises or consists of SEQ ID No. 38 or a variant thereof or a portion thereof.

The ECD of p75NTR has a stalk region (e.g. SEQ ID No. 9, canine stalk region) that is prone to O-glycosylation. Glycosylation in proteins can cause manufacturing difficulties. Thus, in one embodiment, the isolated companion animal ECD may comprise deletions in the stalk region to reduce the number of O-glycosylation sites within the stalk region e.g. to form a truncated stalk region. A truncated stalk region may comprise any number of the amino acids of the stalk region. For example, the stalk region may comprise 1-10, 1-20, 1-30 amino acids. The stalk region may be removed in embodiments of the fusion protein described herein. Thus, a portion of the ECD as used herein may be the ECD without the stalk region and 3′ sequences α-secretase and γ-secretase cleavage sites (e.g. canine sequence SEQ ID No. 34).

Thus, in one embodiment, the isolated companion animal p75NTR is a truncated protein which has the O-glycosylation stalk region removed.

In one embodiment, the isolated companion animal p75NTR protein may be a variant of the wild type protein which comprises one or more amino acid modification compared to the wild type protein. The modification may be a substitution, deletion or addition of an amino acid.

By “variant” or “mutant” herein is meant a polypeptide sequence that differs from that of a wild-type sequence by virtue of at least one amino acid modification. As used herein, a “substitution of an amino acid residue” with another amino acid residue in an amino acid sequence of a protein or polypeptide as described herein, is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence.

Variants of the p75NTR protein or portions thereof as used herein retain the biological function of the wild type protein, that is binding to NGFs.

Amino acid modifications in general refer to and include substitutions, insertions and deletions, with the former being preferred in many cases. The variants of the invention include amino acid substitutions, and they can include any number of further modifications, as long as the function of the protein is still present, as described herein. In one embodiment, from 1, 2, 3, 4, 5, 6, 7, 8, 9 or 10 modifications are generally utilized as often the goal is to alter function with a minimal number of modifications. A variant polypeptide sequence will preferably possess at least about 80%, 85%, 90%, 95% or up to 98% or 99% identity to the wild-type sequences or the parent sequences. It should be noted that depending on the size of the sequence, the percent identity will depend on the number of amino acids. Variants do not include human sequences.

By “protein variant” or “variant protein” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent. The variant polypeptide sequence herein will preferably possess at least about 80% identity with a parent polypeptide sequence, and most preferably at least about 90% identity, more preferably at least about 95% identity. Variants do not include human sequences.

By “parent polypeptide”, “parent protein” as used herein is meant an unmodified polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

By “wild type” or “WT”, “wt” or “native” herein is meant an amino acid sequence or a nucleotide sequence that is found in nature, including allelic variations. A WT protein, polypeptide, Fc domain, immunoglobulin etc. has an amino acid sequence or a nucleotide sequence that has not been intentionally modified.

In another aspect, the invention relates to an isolated nucleic acid encoding a companion animal p75NTR protein or a portion thereof, e.g. the ECD or a portion thereof or a variant. As used herein, a portion of p75NTR or a portion of the ECD of p75NTR includes at least one neurotrophin binding domain. In one embodiment, the companion animal is a dog. For example, the isolated nucleic acid comprises or consists of SEQ ID No. 2, 4, 6, 8 or 37 or a variant thereof or a portion thereof.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature.

In another aspect, the invention relates to a vector, plasmid, transcription, expression cassette or nucleic acid construct comprising a nucleic acid encoding a companion animal p75NTR protein or a portion thereof, e.g. the ECD or portion thereof as described above.

The construct may include a suitable leader sequence. The term leader sequence is used interchangeably with signal sequence. Thus, in some embodiments, the nucleic acid sequence/nucleic acid construct encoding the fusion protein may also comprise a leader sequence. The leader sequence is made as part of the protein and then cleaved off when the protein is secreted. Any suitable leader sequence may be used, including a native immunoglobulin germline leader sequence, such as the endogenous p75 leader of the relevant species (e.g. canine, equine, feline, bovine), the endogenous p75 leader of a different species e.g. human, canine, equine, feline, bovine or a mouse IgG leader or another leader sequences known in the art, e.g. the Campath leader sequence (see U.S. Pat. No. 8,362,208 B2) or an artificial sequence. Such leader sequences can aid in enhancing protein expression.

In another aspect, the invention relates to a host cell comprising a nucleic acid encoding a companion animal p75NTR protein or a portion thereof, e.g. the ECD, or a vector, plasmid, vector, transcription, expression cassette or construct as described above.

Expression vectors of use in the invention may be constructed from a starting vector such as a commercially available vector. After the vector has been constructed and the nucleic acid molecule has been inserted into the proper site of the vector, the completed vector may be inserted into a suitable host cell for amplification and/or polypeptide expression.

The term “vector” means a construct, which is capable of delivering, and in some aspects expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The invention also relates to an isolated recombinant host cell comprising one or more nucleic acid molecule plasmid, vector, transcription or expression cassette as described above. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

The host cell may be eukaryotic or prokaryotic, for example a bacterial, viral, plant, fungal, mammalian or other suitable host cell. In one embodiment, the cell is an E. coli cell. In another embodiment, the cell is a yeast cell. In another embodiment, the cell is a Chinese Hamster Ovary (CHO) cell, HeLa cell or other cell that would be apparent to the skilled person. Mammalian cell lines available as hosts for expression are well known in the art and include, but are not limited to, immortalized cell lines available from the American Type Culture Collection (ATCC) and any cell lines used in an expression system known in the art can be used to make the recombinant polypeptides of the invention.

In general, host cells are transformed with a recombinant expression vector that comprises DNA encoding a protein. Among the host cells that may be employed are prokaryotes, yeast or higher eukaryotic cells. Prokaryotes include gram negative or gram-positive organisms, for example E. coli or bacilli. Higher eukaryotic cells include insect cells and established cell lines of mammalian origin. Examples of suitable mammalian host cell lines include the COS-7 cells, L cells, CI27 cells, 3T3 cells, Chinese hamster ovary (CHO) cells, or their derivatives and related cell lines which grow in serum free media, HeLa cells, BHK cell lines, the CVIIEBNA cell line, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays.

Other suitable host cells include insect cells, using expression systems such as baculovirus in insect cells, plant cells, transgenic plants and transgenic animals, and by viral and nucleic acid vectors.

Alternatively, it is possible to produce the polypeptide in lower eukaryotes such as fungal cell lines and yeast or in prokaryotes such as bacteria. Suitable yeasts include S. cerevisiae, S. pombe, Kluyveromyces strains, Pichia pastoris, Candida, or any yeast strain capable of expressing heterologous polypeptides. Suitable bacterial strains include E. coli, B. subtilis, S. typhimurium, or any bacterial strain capable of expressing heterologous polypeptides. If the protein is made in yeast or bacteria, it may be desirable to modify the product produced therein, for example by phosphorylation or glycosylation of the appropriate sites, in order to obtain a functional product. Such covalent attachments can be accomplished using known chemical or enzymatic methods.

A host cell, when cultured under appropriate conditions, can be used to express a protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

In another aspect, the invention also relates to the use of an isolated companion animal p75NTR protein or a portion thereof as described above in a fusion protein with another moiety, e.g. with a half-life extending moiety as described in more detail below. Therefore, the p75NTR protein or a portion thereof can be provided covalently linked or couple to a half-life extending moiety. Alternatively, it may be provided incorporated in a liposome. The invention further relates to an isolated companion animal p75NTR protein or a portion thereof for use in therapy. Further, there is provided an isolated companion animal p75NTR protein or a portion thereof for use in the treatment of a pain related disease. Such diseases are described in more detail below.

In some embodiments, to improve its pharmacokinetic (PK) properties, the half-life of the p75NTR protein is extended.

### Fusion Proteins

Thus, in another aspect, the invention relates to a fusion protein comprising an isolated companion animal p75NTR protein or portion thereof, e.g. the extracellular domain, as described above and another moiety.

For example, the other moiety may be a half-life extending moiety. Thus, the p75NTR protein or portion thereof is coupled to a half-life extending moiety. As described above, the p75NTR protein or portion thereof may be canine, feline or equine. The p75NTR protein or portion thereof used in the fusion protein may thus comprise or consist of a sequence selected from SEQ ID NO. 1, 3, 5, 7, 34, 36 or 38 or a portion or a variant thereof. In one embodiment of the canine p75NTR protein or portion, the stalk region (e.g. SEQ ID NO. 9) is removed.

Half-life extending moieties have been described. For example, the half-life extending moiety may be selected from the following non-limiting list: a companion animal immunoglobulin Fc domain, polyethylene glycol (PEG), PEG derivatives, simple lipids, lipid dicarboxylic acids, lipids with additional moieties, companion animal serum albumin binders, e.g. small-molecule binders or antibodies/antibody fragments that bind companion animal serum albumin, companion animal serum albumin, or streptococcal protein G's albumin-binding domain (ABD). Examples of lipids include glucagon-like peptide 1 (GLP-1), the analogs GLP-1 liraglutide and semaglutide or cholesterol. Advantageously, using an immunoglobulin Fc domain facilitates purification of the protein. In particular, Fc binding to Protein A can be used in purification procedures. The presence of an immunoglobulin Fc domain can also stabilise the overall folding of the fusion protein as well as extending its half-life.

In one embodiment, where the half-life extending moiety is a companion animal Fc domain, companion animal serum albumin binder or companion animal serum albumin, the p75NTR protein or portion and half-life extending moiety are from/specific to the same companion animal. For example, in one embodiment, the half-life extending moiety is a companion animal Fc domain of the corresponding companion animal. For example, if the p75NTR protein or portion thereof, e.g. the extracellular domain is canine, the Fc domain is canine. If the p75NTR protein or portion thereof, e.g. the extracellular domain is feline, the Fc domain is feline. If the p75NTR protein or portion thereof, e.g. the extracellular domain is equine, the Fc domain is equine. If the p75NTR protein or portion thereof, e.g. the extracellular domain is bovine, the Fc domain is bovine.

However, given the high sequence similarity between companion animal p75 protein, in another embodiment, where the half-life extending moiety is a companion animal Fc domain, companion animal serum albumin binder or companion animal serum albumin, the p75NTR protein or portion and half-life extending moiety are not from/specific to the same companion animal. For example, in one embodiment, the half-life extending moiety is the companion animal Fc domain of the corresponding companion animal, but the p75 protein or portion thereof is that of a different companion animal. For example, for treatment of dogs, if the Fc domain is canine, the p75NTR protein or portion thereof, e.g. the extracellular domain is may be from a different animal, e.g. cat, horse or cow. For example, for treatment of cats, if the Fc domain is feline, the p75NTR protein or portion thereof, e.g. the extracellular domain is may be from a different animal, e.g. dog, cow or horse. For example, for treatment of cats, if the Fc domain is equine, the p75NTR protein or portion thereof, e.g. the extracellular domain is may be from a different animal, e.g. cat, cow dog. In yet another embodiment, human p75 or a portion thereof fused to companion animal Fc can be used.

The companion animal serum albumin binder, e.g. antibody or fragment thereof, may be canine or caninized, feline of felinized, equine or equinized. The companion animal serum albumin binder may bind to canine, feline or equine serum albumin.

In one embodiment, the half-life extending moiety is a wild type or variant Fc domain. The term variant is as defined above. For example, an Fc domain variant may have modified half-life compared to the wild type Fc domain. In one embodiment, the Fc domain is a canine Fc domain, that is a wild type domain or a variant thereof. Variant Fc domains are described, for example in WO2020/142625.

By “Fc” or “Fc region” or “Fc domain” as used herein is meant the polypeptide comprising the constant region of an antibody excluding the first constant region immunoglobulin domain (CH1) and, in some cases, part of the hinge. In one embodiment, the Fc domain includes constant region immunoglobulin domains CH2, CH3 and the hinge region between CH1 and CH2 or part of the hinge region.

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). Exemplary amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Amino acid sequences for IgG-A, IgG-B, IgG-C and IgG-D as used by the inventors and according to the aspects and embodiments of the invention are provided as SEQ ID Nos. 15, 16, 17, 18).

In human, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains CH2 and CH3 and the lower hinge region between CH1 and CH2. Although the boundaries of the Fc region may vary, the human IgG heavy chain Fc region is usually defined to include residues C226 or P230 to its carboxyl-terminus, wherein the numbering is according to the EU index as in Kabat.

Fc as used herein may refer to the Fc region in isolation, or this region in the context of an Fc fusion (“fusion composition” or “fusion construct”), as described herein. Fc domains include all or part of an Fc region; that is, N- or C-terminal sequences may be removed from wild-type or variant Fc domains, as long as this does not affect function.

Briefly, IgG functions are generally achieved via interaction between the Fc region of the Ig and an Fcγ receptor (FcγR) or another binding molecule, sometimes on an effector cell. This can trigger the effector cells to kill target cells to which the antibodies are bound through their variable (V) regions. Also, antibodies directed against soluble antigens might form immune complexes which are targeted to FcγRs which result in the uptake (opsonisation) of the immune complexes or in the triggering of the effector cells and the release of cytokines.

By “Fc gamma receptor”, “FcγR” or “FcgammaR” as used herein is meant any member of the family of proteins that bind the IgG antibody Fc region and is encoded by an FcγR gene.

In humans, three classes of FcγR have been characterised, although the situation is further complicated by the occurrence of multiple receptor forms. The three classes are:
(i) FcγRI (CD64) including isoforms FcγRIa, FcγRIb, and FcγRIc binds monomeric IgG with high affinity and is expressed on macrophages, monocytes, and sometimes neutrophils and eosinophils;
(ii) FcγRII (CD32) binds complexed IgG with medium to low affinity and is widely expressed. These receptors can be divided into two important types, FcγRIIa and FcγRIIb. The ‘a’ form of the receptor is found on many cells involved in killing (e.g. macrophages, monocytes, neutrophils) and seems able to activate the killing process and occurs as two alternative alleles. The ‘b’ form seems to play a role in inhibitory processes and is found on B-cells, macrophages and on mast cells and eosinophils. On B-cells it seems to function to suppress further immunoglobulin production and isotype switching to for example, the IgE class. On macrophages, the b form acts to inhibit phagocytosis as mediated through FcγRIIa. On eosinophils and mast cells the b form may help to suppress activation of these cells through IgE binding to its separate receptor and
(iii) FcγRIII (CD16) binds IgG with medium to low affinity and exists as two types. FcγRIIIa is found on NK cells, macrophages, eosinophils and some monocytes and T cells and mediates ADCC. FcγRIIIb is highly expressed on neutrophils. Both types have different allotypic forms.

Canine Fc receptors are described in Bergeron et al L. M. Bergeron et al.; Veterinary Immunology and Immunopathology 157 (2014) 31-41. Canine has RI, RIIb, RIII, but not Riia.

As well as binding to FcγRs, IgG antibodies can activate complement and this can also result in cell lysis, opsonisation or cytokine release and inflammation. The Fc region also mediates such properties as the transportation of IgGs to the neonate (via the so-called “FcRn”), increased half-life (also believed to be effected via an FcRn-type receptor) and self-aggregation. The Fc-region is also responsible for the interaction with protein A and protein G (which interaction appears to be analogous to the binding of FcRn).

By “effector function” as used herein is meant a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP) and complement-dependent cytotoxicity (CDC).

In one embodiment, the companion animal p75NTR protein or portion thereof, e.g. the extracellular domain, and the other moiety are linked with a linker moiety or otherwise conjugated, attached or covalently or non-covalently linked. Suitable linkers are known to the skilled person. For example, the linker is a peptide linker, such as a glycine and/or alanine and/or threonine and/or serine-rich linker e.g. a glycine-serine linker, such as (G₄S)ₙ wherein n is 1 to 4.

In another embodiment, the linker can be cleavable.

In one embodiment, the companion animal p75NTR protein or portion thereof comprises or consists of a canine p75NTR ECD or portion thereof. In one embodiment, the ECD comprises of consists of SEQ No. 7 or a variant thereof.

Thus, in one embodiment, the invention relates to a fusion protein comprising a canine p75NTR ECD linked to a canine Fc domain. In one embodiment, the ECD comprises of consists of SEQ No. 7 or a variant thereof.

In one embodiment, the fusion protein of the present invention preferably binds to any one or more of NGF, BDNF, NT3 or NT4/5 with a binding affinity (Kd) of between about 1 pM to about 100 nM. In some preferred embodiments, the binding affinity (Kd) is between about 5 pM and any of about 10 pM, 20 pM, 40 pM, 50 pM 100 pM, 0.2 nM, 0.5 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 15 nM, 20 nM, 25 nM, 30 nM, 35 nM, 40 nM, 45 nM, 50 nM or 100 nM as measured in an in vitro binding assay for NGF, BDNF, NT3 or NT4/5 such as described herein. Subnanomolar range is preferred.

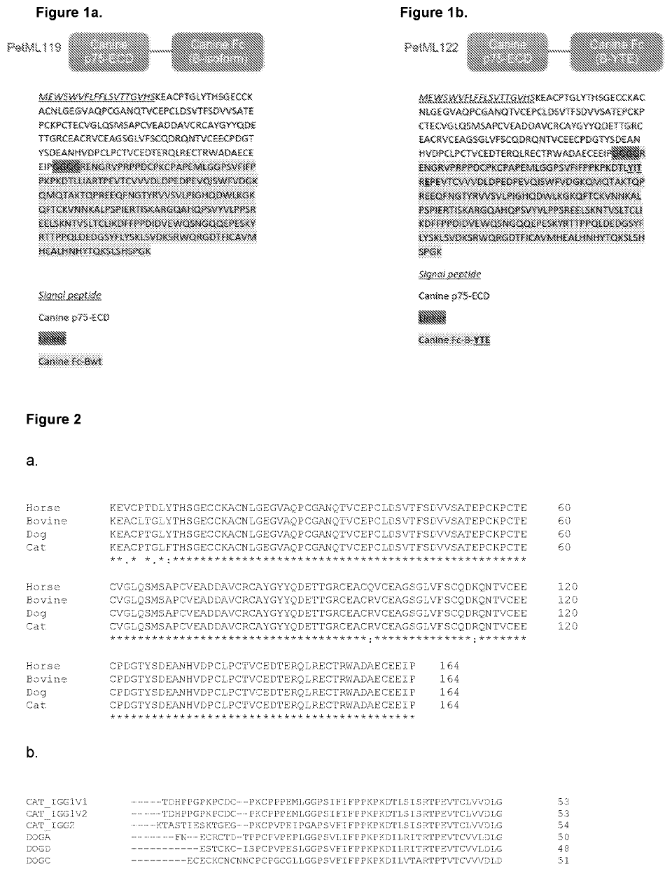

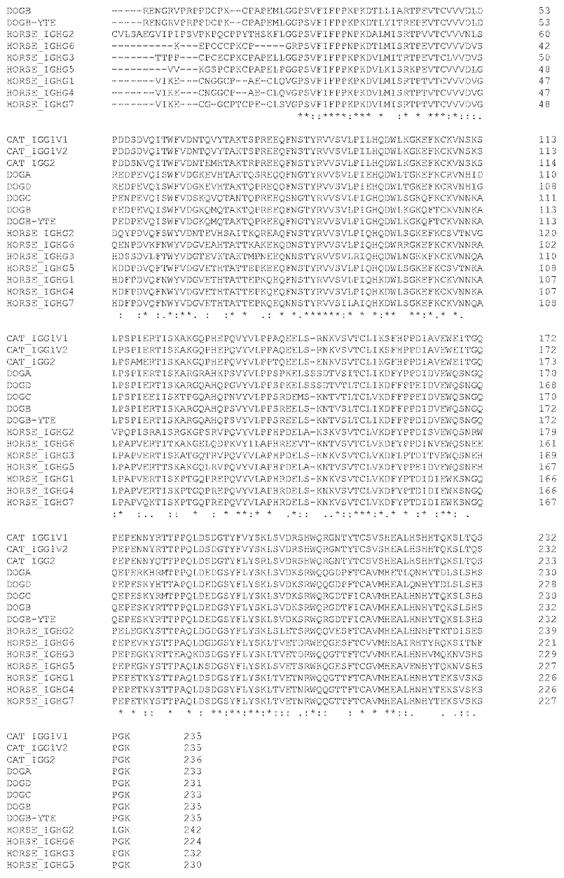

In one embodiment, the fusion protein comprises or consists of SEQ ID NO. 11 or 13 or a variant thereof. These fusion proteins include the p75 ECD operably linked to a canine Fc domain as shown in FIG. 1. The Fc domain in the construct of SEQ ID NO. 11 is a wild type canine Fc domain. The Fc domain in the construct of SEQ ID NO. 13 is a variant canine Fc domain which has been modified to increase half-life. In this domain, the mutation YTE has been introduced at residues Y252-T254—of the wt Fc domain using EU numbering.

In another embodiment, the fusion protein comprises or consists of SEQ ID NO. 39. Such a fusion proteins includes the p75 ECD operably linked to a feline Fc domain. The Fc domain in the construct of SEQ ID NO. 39 is a wild type feline Fc domain.

Therefore, modified companion animal Fc domains that include this mutation, e.g. canine, feline or equine Fc domains, can be used in the fusion proteins of the invention. A skilled person would know that any other known mutations that increase half-life could also be introduced in the Fc domain.

According to the present invention, the fusion proteins demonstrate advantageous biological properties including improved solubility, stability and/or improved serum half-life. Improved solubility and stability is, for example, demonstrated in Examples 4 and 9. These examples show that the described molecules are very stable in both temperature and chemical stress, showing unfolding only when incubated at temperature higher than 70° C. with no aggregation up to 95° C. with Tm1 around 67° C. Improved half-life is, for example, demonstrated in Examples 11 and 12. Improved half-life allows for less frequent dosing (a single administration in comparison to existing treatments where daily administration is required).

This effect is demonstrated whilst showing strong analgesic effects. In one embodiment, the fusion protein of the invention has a half-life in-vivo of about or more than any one of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 hours+/−1 hour, further preferably the p75NTR(NBP)-Fc fusion protein of the invention has a half-life in-vivo of about or more than 24 hours.

In another embodiment, the fusion protein of the invention has a half-life in-vitro of about or more than any one of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 days+/−1 day, further the fusion protein has a half-life in-vitro of about or more than 6 days or more than 1 month. In one embodiment, the half life is 14 days.

According to the foregoing preferred embodiments, the in-vivo half-life can be the half-life in rat or in the corresponding companion animal, e.g. in a dog.

Fusion proteins of the invention can operate at a very low dose, but are highly efficacious. This is, for example, demonstrated in Examples 11 and 12 where administration of PetML119 and PetML122 in rats and dogs outcompete existing treatments.

According to the present invention, the fusion proteins display a good safety profile. This is, for example, demonstrated in Example 12, where animals maintain normal body weight and haematological parameters and do not generate anti-Drug Antibodies after administration of fusion proteins. The maintenance of normal body weight after administration is, for example, also demonstrated in Example 11. This is in contrast to the administration of dexamethasone which decreased body weight over time.

In another aspect, the invention relates to an isolated nucleic acid encoding a fusion protein as described above, for example a fusion protein encoding SEQ ID NO. 11, 13 or 39. In one embodiment, the nucleic acid is selected from SEQ ID NO. 12, 14 or 40.

In another aspect, the invention relates to a vector, plasmid, vector, transcription, expression cassette or construct comprising a nucleic acid described above.

In another aspect, the invention relates to a host cell comprising a nucleic acid vector, plasmid, vector, transcription, expression cassette or construct as described above. Suitable host cells are described elsewhere herein.

In another embodiment, the P75NTR protein, portion thereof or fusion protein is labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

In another aspect, there is provided a pharmaceutical composition comprising a p75NTR protein or portion thereof or a fusion protein of the invention. The fusion protein or pharmaceutical composition described herein can be administered by any convenient route, including but not limited to oral, topical, parenteral, sublingual, rectal, vaginal, ocular, intranasal, pulmonary, intradermal, intravitrial, intratumoural, intramuscular, intraperitoneal, intravenous, subcutaneous, intracerebral, transdermal, transmucosal, by inhalation, or topical, particularly to the ears, nose, eyes, or skin or by inhalation. In another embodiment, delivery is of the nucleic acid encoding the drug, e.g. a nucleic acid encoding the molecule of the invention is delivered.

Parenteral administration includes, for example, intravenous, intramuscular, intraarterial, intraperitoneal, intranasal, rectal, intravesical, intradermal, topical, intra-articular or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the polypeptide of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition can be in the form of a liquid, e.g., a solution, syrup, solution, emulsion or suspension. The liquid can be useful for oral administration or for delivery by injection, infusion (e.g., IV infusion) or sub-cutaneous.

When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e.g. a gelatin capsule), it can contain, in addition to materials of the above type, a liquid carrier such as polyethylene glycol, cyclodextrin or a fatty oil.

When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the polypeptide, Fc domain or pharmaceutical composition described herein that is effective/active in the treatment of a particular disease or condition will depend on the nature of the disease or condition and can be determined by standard clinical techniques. In addition, in vitro or in vivo assays can optionally be employed to help identify optimal dosage ranges. The precise dose to be employed in the compositions will also depend on the route of administration, and the seriousness of the disease or disease, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of a polypeptide of the present invention by weight of the composition. When intended for oral administration, this amount can be varied to range from about 0.1% to about 80% by weight of the composition. Preferred oral compositions can comprise from about 4% to about 50% of the polypeptide of the present invention by weight of the composition.

Compositions can be prepared so that a parenteral dosage unit contains from about 0.01% to about 2% by weight of the polypeptide of the present invention.

For administration by injection, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks or more.

Treatment can for example be once a month or bi-monthly. This is advantageous over daily administration as this improves compliance and minimises stress to the animal.

As used herein, “treat”, “treating” or “treatment” means inhibiting or relieving a disease or disease. For example, treatment can include a postponement of development of the symptoms associated with a disease or disease, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., canine patients, being treated. Many medical treatments are effective for some, but not all, patients that undergo the treatment.

The term “subject” or “patient” refers to an animal which is the object of treatment, observation, or experiment, suitably a companion animal, specifically a canine or a feline.

In another aspect, the invention relates to the use of a p75NTR protein or portion thereof, fusion protein or pharmaceutical composition described herein in the treatment or prevention of a disease. In another aspect, the disclosure relates to the use of a polypeptide, Fc domain or pharmaceutical composition described herein in the manufacture of a medicament for the treatment or prevention of a disease as listed herein. The invention further relates to a method of treating a disease in a subject comprising an effective amount of the polypeptide, Fc domain or pharmaceutical composition as described herein to said subject.

For example, the disease is a NGF related disorder.

In one embodiment, the NGF related disorder is selected from the group consisting of: cardiovascular diseases, atherosclerosis, obesity, type 2 diabetes, metabolic syndrome, pain and inflammation. In one embodiment, the NGF related disorder comprises pain. In one embodiment, the pharmaceutical composition is used in the treatment of pain. In one embodiment, the pharmaceutical composition is used for the treatment of a pain and the type of pain is selected from osteoarthritis pain, rheumatoid arthritis pain, surgical and postsurgical pain, incisional pain, general inflammatory pain, cancer pain, pain from trauma, neuropathic pain, neuralgia, diabetic neuropathy pain, pain associated with rheumatic diseases, pain associated with musculoskeletal diseases, visceral pain, and gastrointestinal pain. In one embodiment, the pain comprises osteoarthritis pain. In one embodiment, the pain comprises surgical and post-surgical pain. In one embodiment, the pain comprises cancer pain.

In one or more embodiments, the p75NTR protein or portion thereof, fusion protein or pharmaceutical composition of the invention is for use in a canine. In one or more embodiments, the p75NTR protein or portion thereof, fusion protein or pharmaceutical composition of the invention is for use in felines. In one or more embodiments, the p75NTR protein or portion thereof, fusion protein or pharmaceutical composition of the invention is for use in equine.

In one embodiment, the p75NTR protein or portion thereof, fusion protein or pharmaceutical composition of the invention is administered together with one or more therapeutic agent, for example a therapeutic agent to treat pain.

The polypeptide, the p75NTR protein or portion thereof, fusion protein or pharmaceutical composition may be administered at the same time or at a different time as the other therapy or therapeutic compound or therapy, e.g., simultaneously, separately or sequentially.

The invention also provides an in vitro or in vivo method for inhibiting NGF activity in a companion animal comprising administering the p75NTR protein or portion thereof, fusion protein or pharmaceutical composition of the invention.

In one or more aspects, the present invention provides a method of producing the fusion protein of the invention by culturing the host cell of the invention under conditions that result in production of the fusion protein and subsequently isolating the fusion protein from the host cell or culture medium of the host cell.

In another aspect, the invention provides a kit for the treatment or prevention of a disease, diagnosis, prognosis or monitoring disease comprising a the p75NTR protein or portion thereof, fusion protein or pharmaceutical composition of the invention of the invention. Such a kit may contain other components, packaging and/or instructions.

The invention in another aspect provides a the p75NTR protein or portion thereof, fusion protein or pharmaceutical composition of the invention packaged in lyophilized form or packaged in an aqueous medium.

In another aspect, a p75NTR protein or portion thereof, fusion protein or pharmaceutical composition of the invention as described herein is used for non-therapeutic purposes, such as diagnostic tests and assays. Thus, the present invention also provides the above p75NTR proteins and fusion proteins for use in diagnostic methods for detecting NGF in species, particularly canines and felines, known to be or suspected of having an NGF related disorder. Methods for detecting NGF in species, particularly canines and felines, known to be or suspected of having an NGF related disorder may include exposing a sample from the animal to a labelled protein of the invention and detecting said labelled protein. may be used to quantitatively or qualitatively detect the NGF in a sample or to detect presence of cells that express the NGF.

Further aspects and embodiments of the invention will be apparent to those skilled in the art given the present disclosure including the following experimental exemplification.

Unless otherwise defined herein, scientific and technical terms used in connection with the present disclosure shall have the meanings that are commonly understood by those of ordinary skill in the art.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including any references to gene accession numbers and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further described in the non-limiting examples.

### Examples

### Methods:

### 1. Protein Constructs and CHO-s Transfection/Expression (FIG. 1)

For protein production, DNA constructs were generated to encode chimeric Fc fusion protein comprising selected canine IgG constant regions (between hinge and C-terminus) fused to the extracellular domain of canine p75 lacking the predicted O-glycosylation rich stalk region and the α- and γ-secretase sites. The amino acid sequences for PetML119 (SEQ ID NO: 11) and PetML122 (SEQ ID NO: 13) are given in FIG. 1. An alignment of canine, feline, bovine and equine p75 sequence portions is provided in FIG. 2.

Both the canine IgG-B Fc domain and the p75 extracellular domain (res 31-194 from UniProtKB—J9PAM0) were codon optimised and synthesised by GeneArt (Thermo Fisher). Both genes were PCR amplified using Q5 high fidelity DNA polymerase (using specific primers including overlapping regions to allow assembly) and assembled into mammalian expression vector PetML119 (Fc-Bwt) and PetML122 (Fc-B-YTE with the mutated residues Y252-T254-E256 using EU numbering) using NEBuilder HIFI DNA Assembly (New England Biolabs). In the expression vector, the fusion protein chain and the antibiotic resistant gene expression units are flanked by DNA transposon piggyBac terminal inverted repeats to mediate stable integration into host cells in the presence of piggyBac transposase. Both expression vectors also contain puromycin resistant cassette which is located within the piggyBac terminal repeats to facilitate selection for stable integration. To generate stable expression cell lines, PetML119 or PetMI122 was transfected into a suitable mammalian cell line such as CHO cells together with PiggyBac transposase followed by puromycin selection at 10-30 μg/ml for at least 8-10 days. For fusion protein production, 1×10⁶/mL selected CHO cells are seeded in 800 mL culture media (F17+4 mM I-Gln+0.3% P188+1:500 ACA) and incubated at 32° C., 8% CO₂ with shaking at 130 rpm. 2% HyClone Cell Boost 7a supplement+0.2% HyClone Cell Boost 7b supplement 2 mM glucose is added to the media daily from the 4^{th} day of overproduction. Culture supernatants are collected on day 10 and the protein concentration is determined using surface plasmon resonance using protein A chip (Biacore 8K, Cytiva Life Sciences). Typically, PetML119/122 showed peak of expression at 10 day in production reaching around 70 mg/L.

### 2. PetML119-122 Purification

Cell suspensions from PetML119 and PetML122 stable transfected clones, cultured as described for at least 7 days, were filtered using 0.22 um filters after being incubated for 10 minutes with Sartoclear Dynamics® Lab V (SDLV-0500-20C-E). Cleared supernatants have been loaded into Mabselect sure LX prepacked 20 mL column (17547402), pre-equilibrated with PBS. Column was washed with 40 mLs of PBS (2CV) and then fusion proteins were eluted using gradient (0-100% in 2CV) of 0.1M Glycine pH2.7. Fractionations containing fusion proteins have been pooled together and neutralised with 100 mM TRIS pH8 (final concentration).

Neutralised fusion protein pooled fractions were concentrated till 5 mL and loaded into PBS pre-equilibrated HiLoad 16/600 Superdex 200 μg (28989335) as second step purification. Monomeric fractions (based on previously analysed protein standards' retention times) were pooled and protein concentration was assessed using NanoDrop™ One (Thermo Scientific™).

Around 30 mg/L of purified product was obtained following the above mentioned protocol.

PetML119 showed 34 mg/L final recovery after two step purification (with acceptable Endotoxin and HCP using standard protocols and buffers).

PetML122 showed 28 mg/L final recovery after two step purification (with acceptable Endotoxin and HCP using standard protocols and buffers)

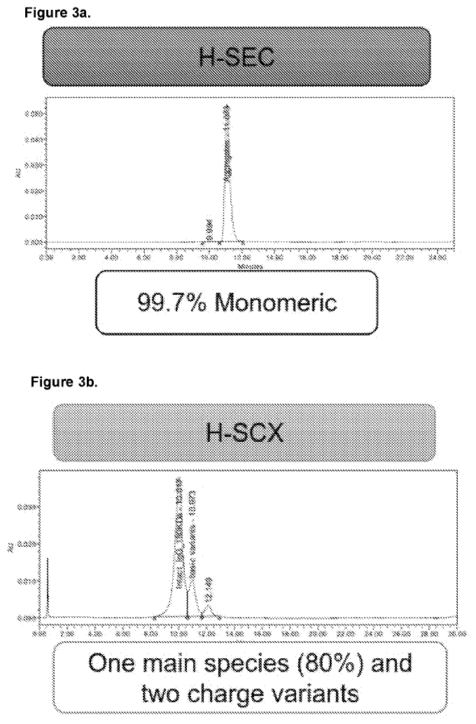

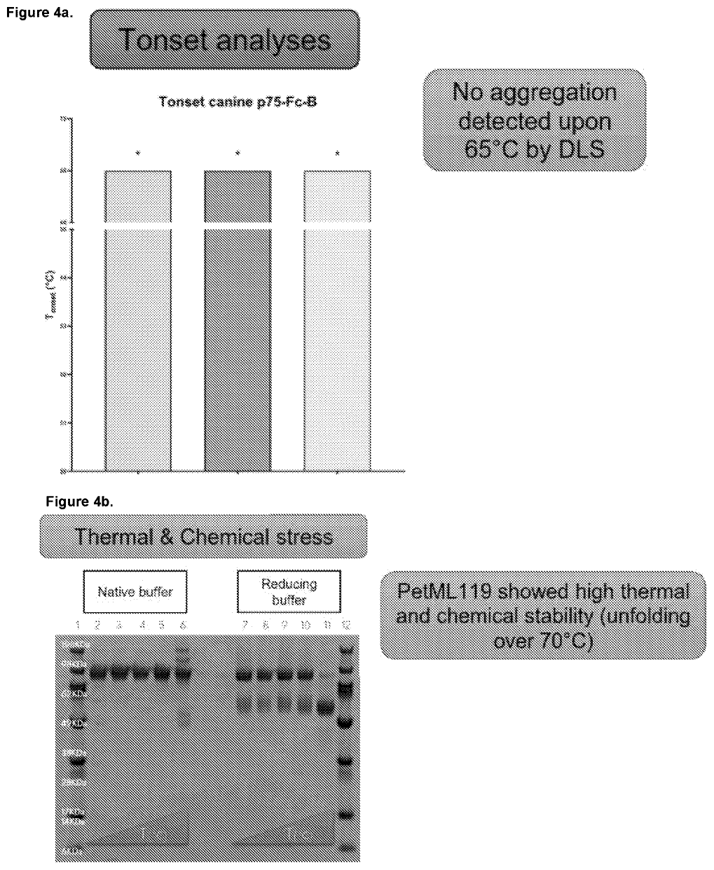

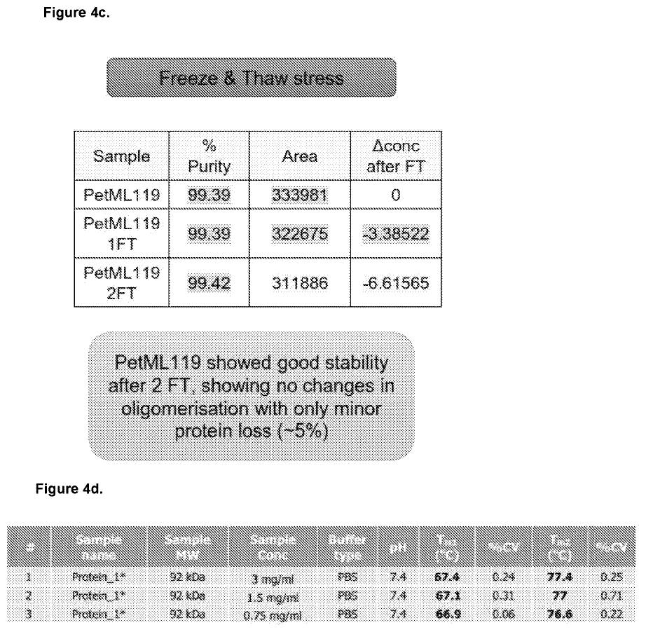

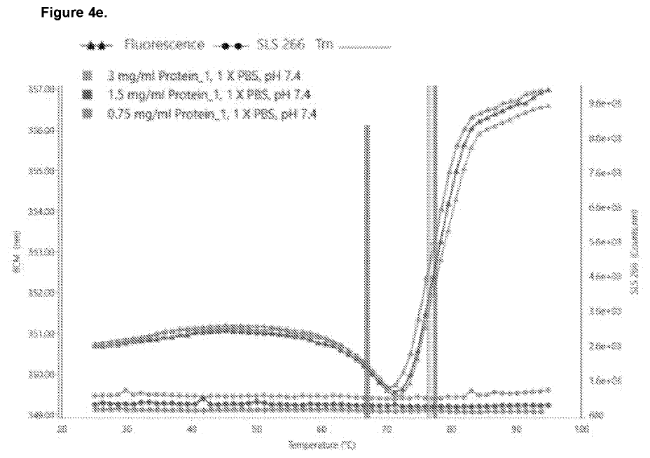

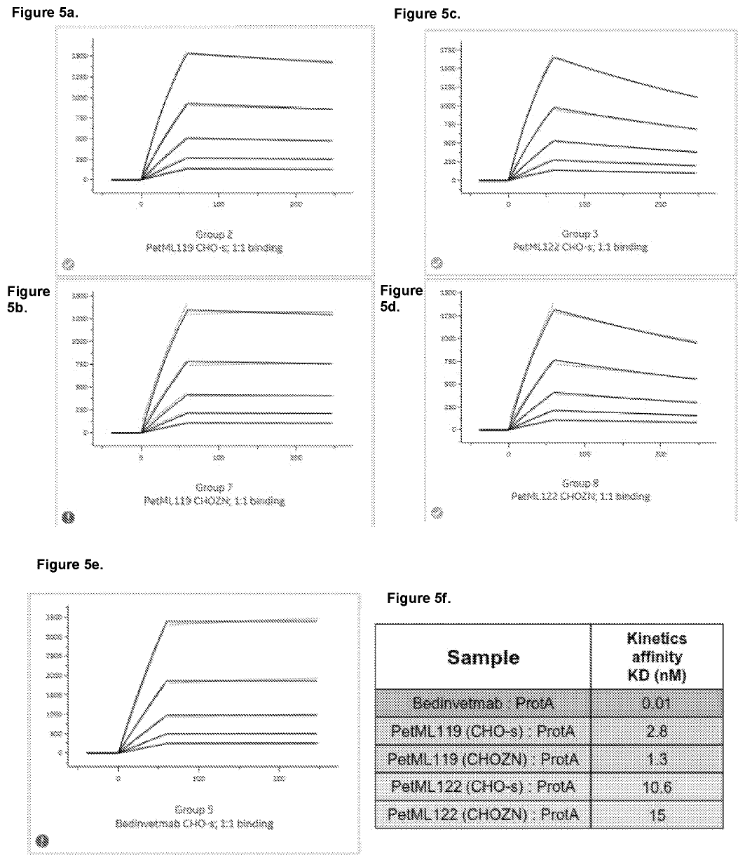

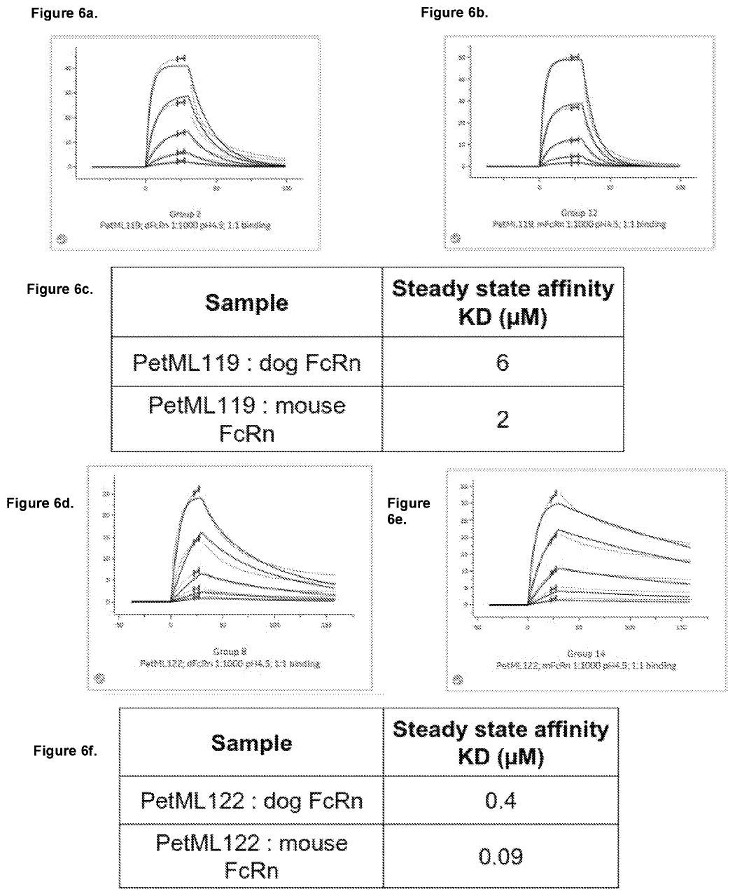

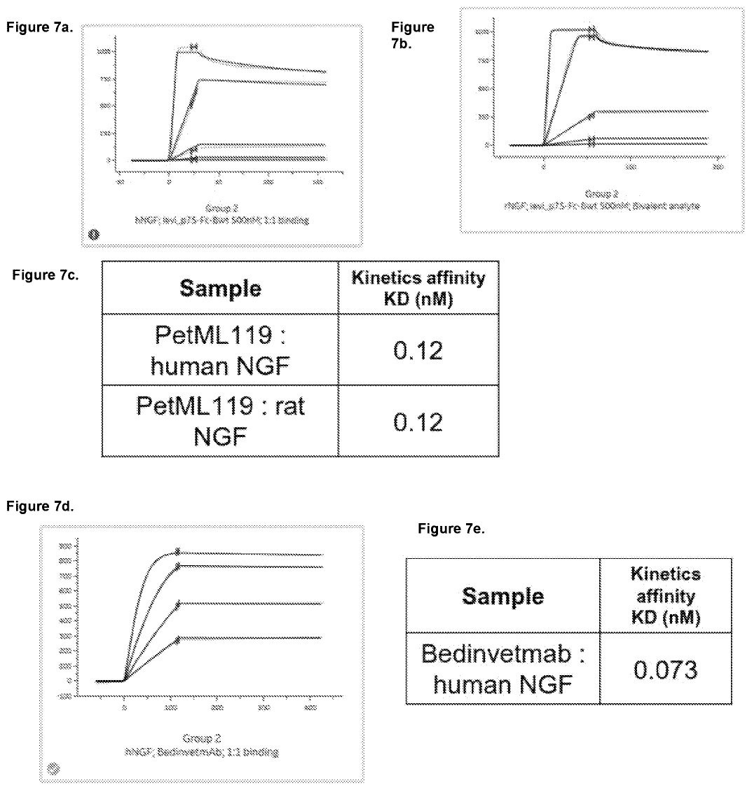

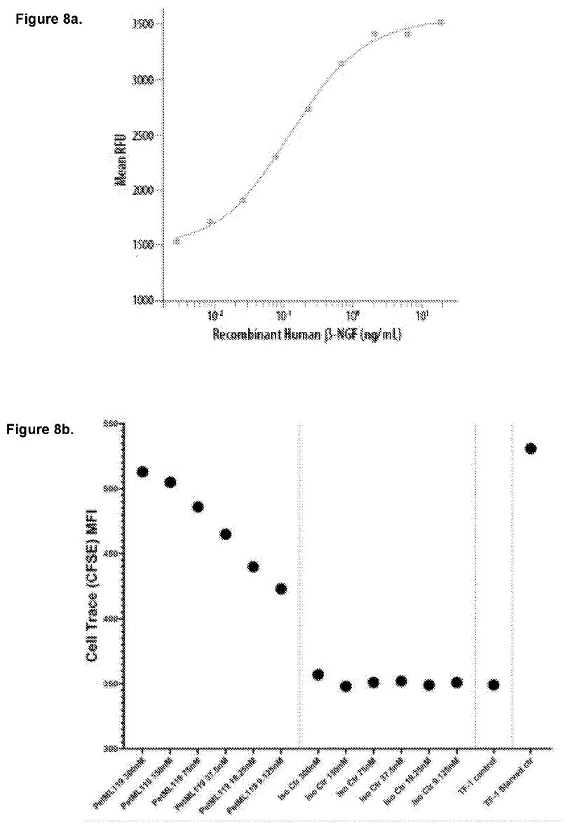

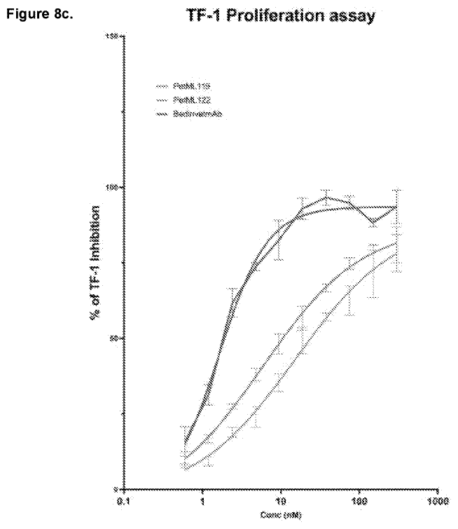

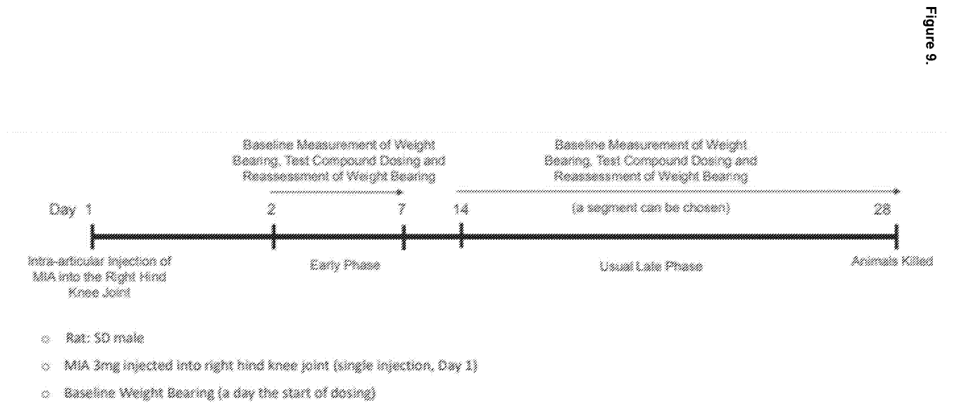

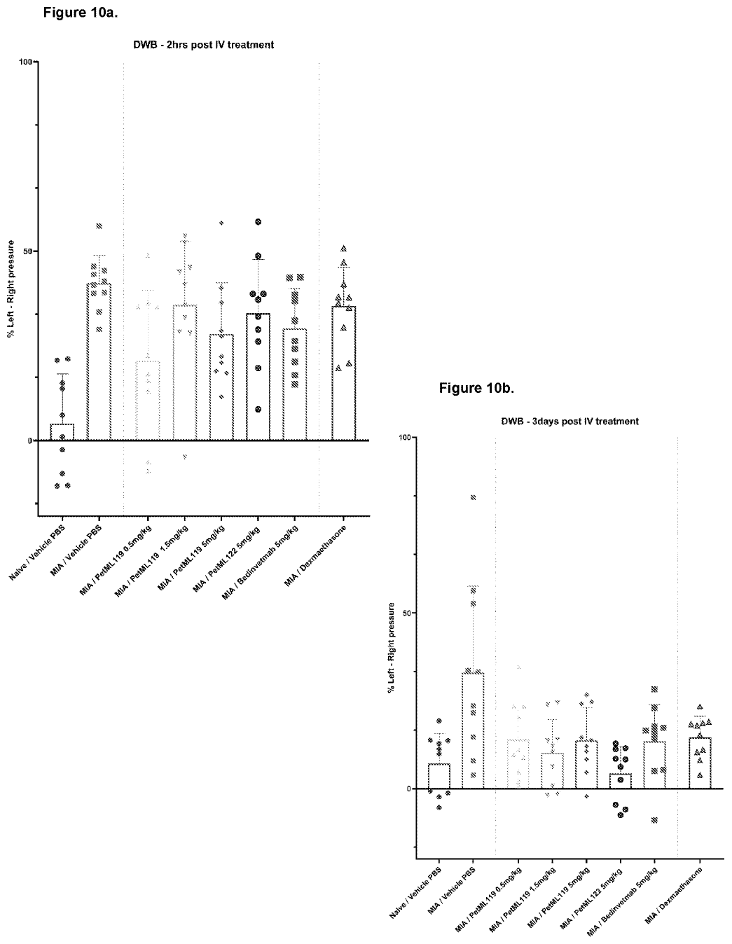

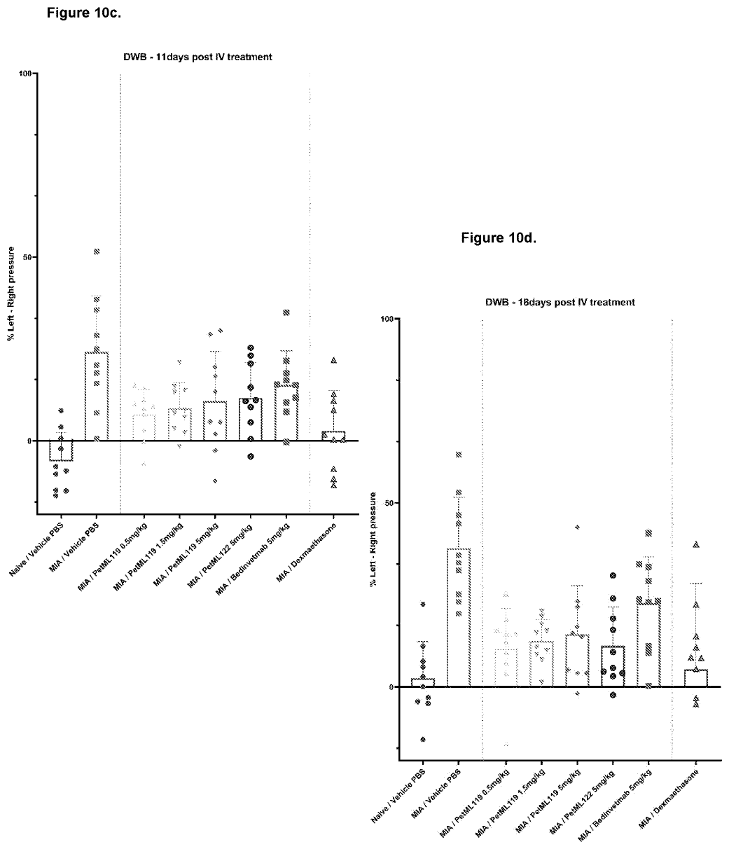

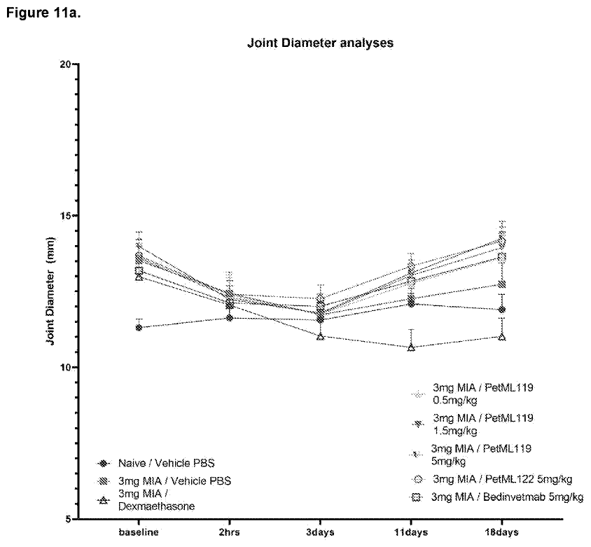

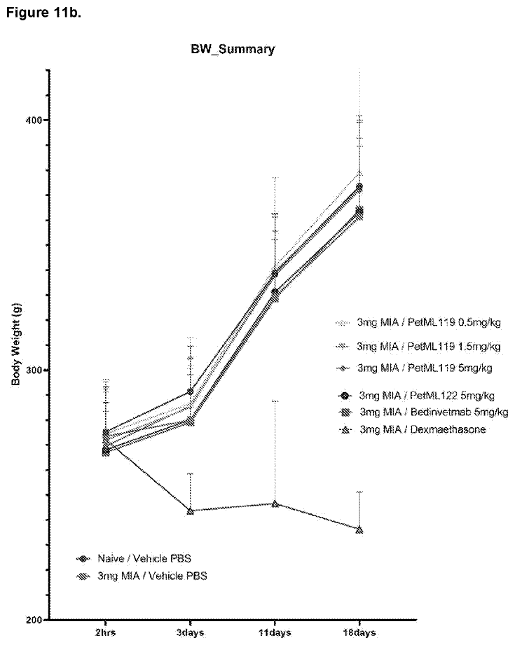

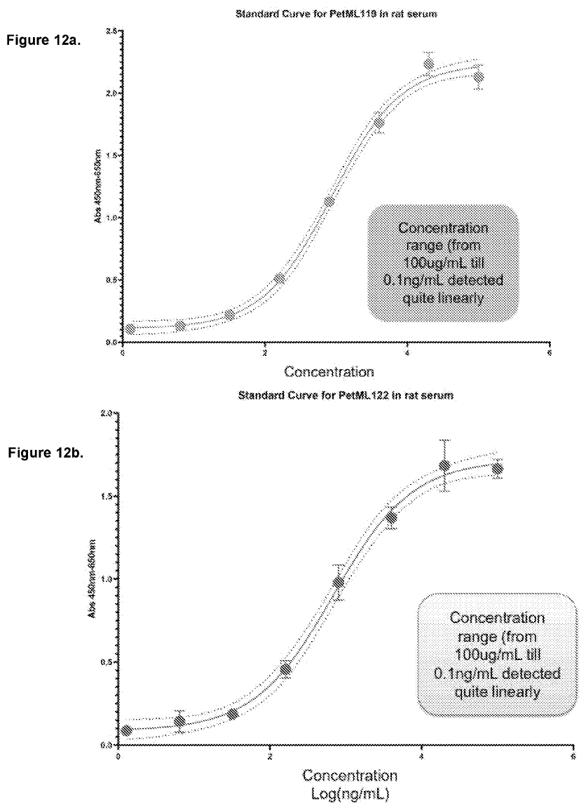

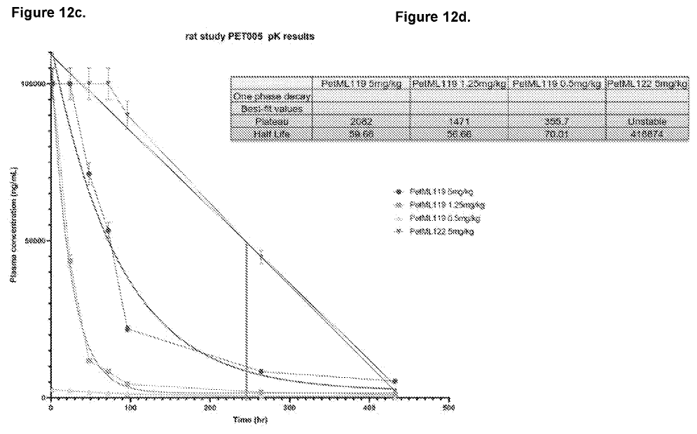

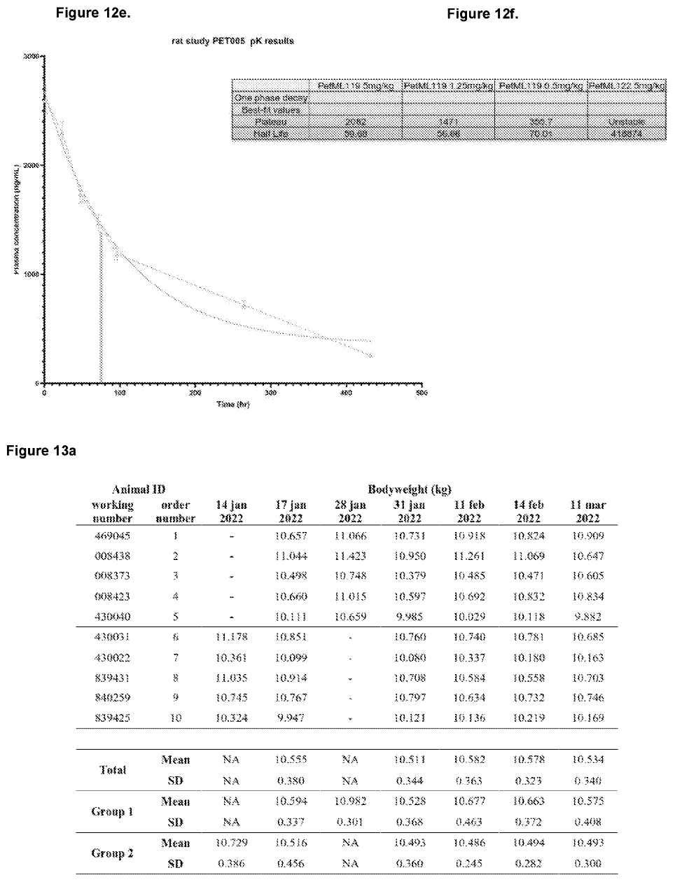

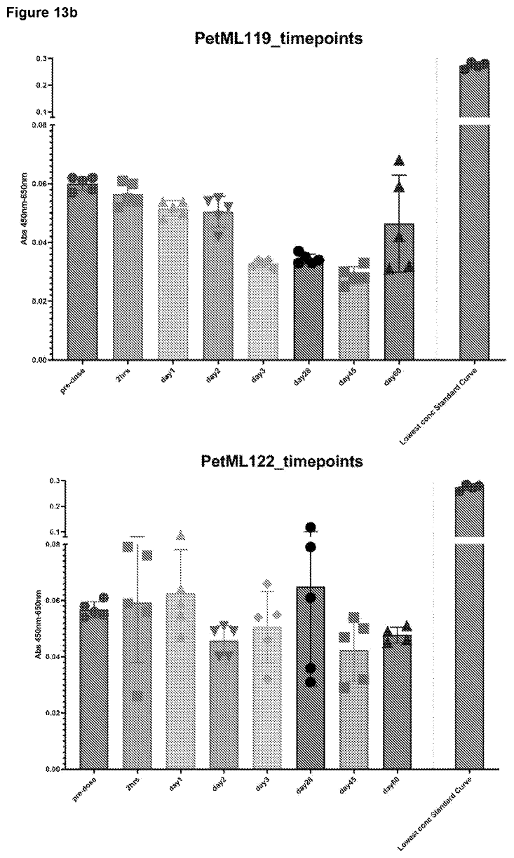

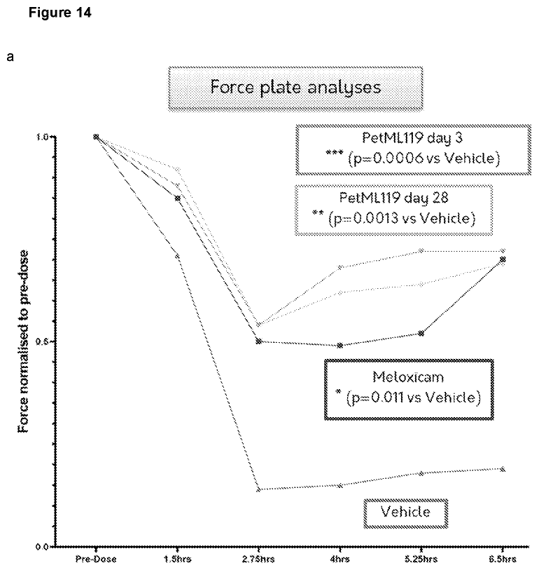

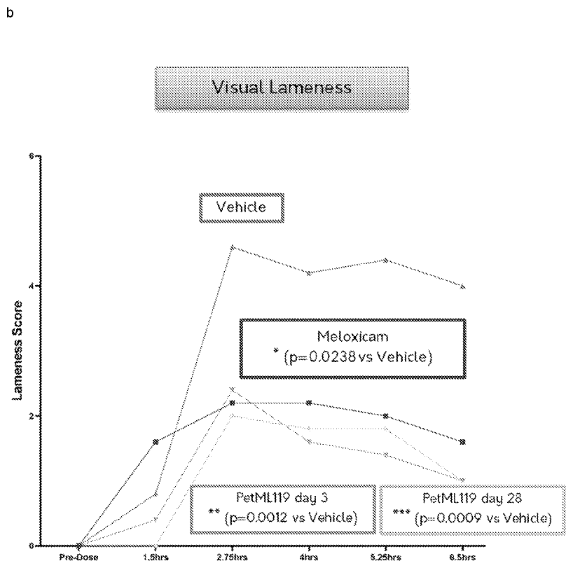

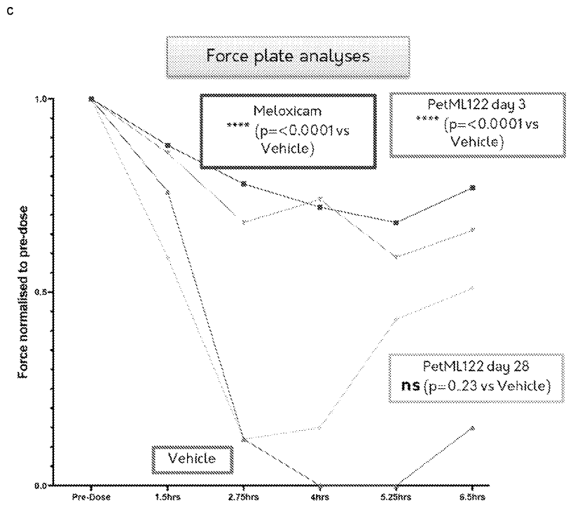

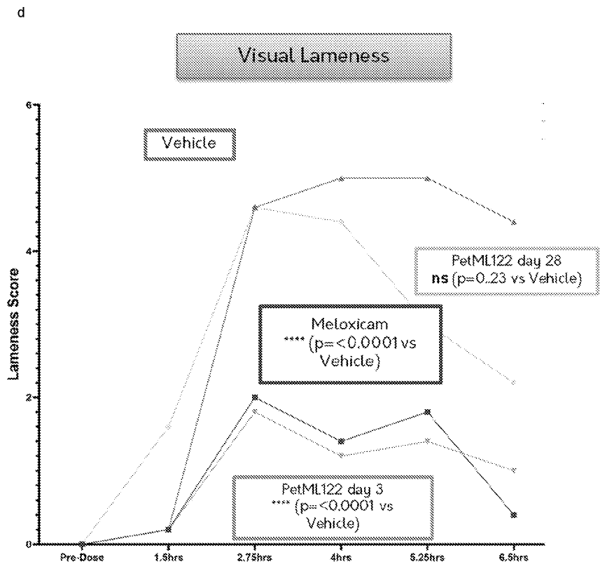

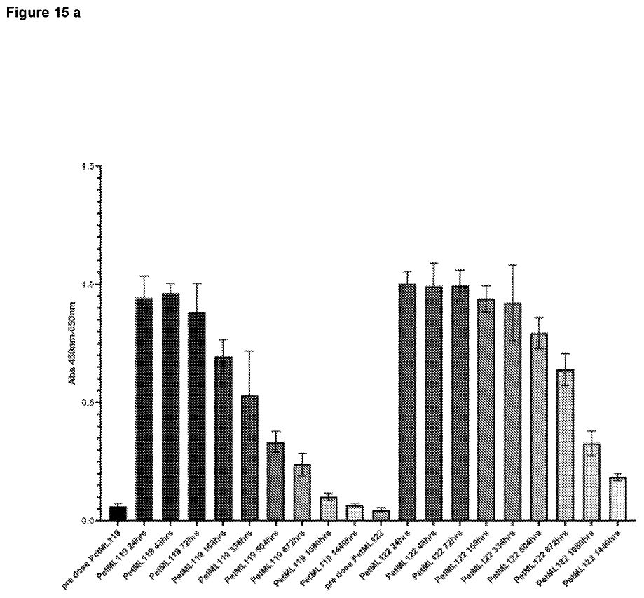

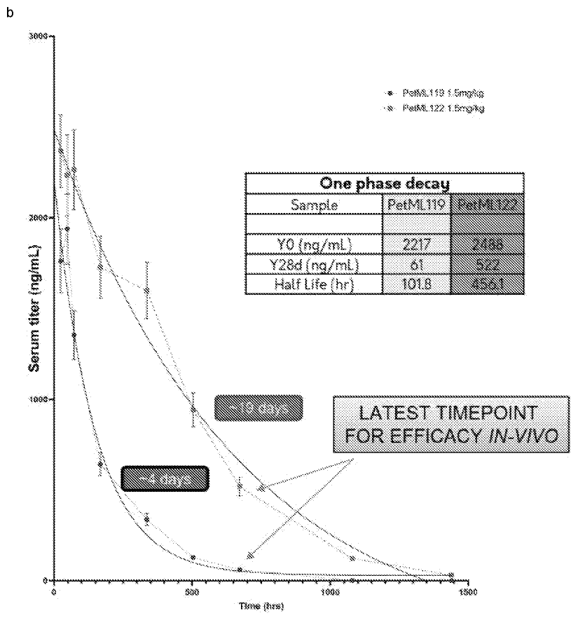

### 3. HPLC Analytical Chromatography (FIG. 3)

Purified material purity was assessed using both Size Exclusion Chromatography (SEC), for oligomerisation analyses, and cation exchange chromatography (SCX) for charge variants analyses.

HPLC-SEC chromatography (column: BioResolve SEC mAb 200A, 2.5 um column WATERS) was performed using ACQUITY H-class Bio from WATERS using PBS as mobile phase with isocratic 0.575 mL/min flow rate.

HPLC-SCX chromatography (column: BioResolve SCX mAb Column, 3 μm, 4.6 mm×100 mm) was performed using ACQUITY H-class Bio from WATERS using MES pH5 as mobile phase with salt gradient used to separate charge variants at 0.9 mL/min flow rate. 10 uL of each sample were injected into both H-SEC/H-SCX using the above-mentioned protocol.

Percentage of monomeric species and Area (indicative of protein concentration) were determined for each molecule.

Both PetML119/122 showed very high purity (more than 99%) by HSEC and few charge variants (potentially corresponding to different glycoforms) were observed by HSCX.

### 4. Stress Tests (FIG. 4)

### Freeze and Thaw Test:

50 μL aliquots of PetML119 at 5 mg/mL in PBS were prepared and stored at either 4° C. (1 aliquot per molecule) or −80° C. (3 aliquots per molecules) in sealed PCR tubes. −80° C. aliquots were thawed at room temperature in 5 minutes and stored back at −80° C. leaving one aliquot out (1 freeze and thaw). This have been repeated two more times (2 and 3 freeze and thaw cycles) and then 10 μL of each sample (including 4° C. controls) were injected into H-SEC using same method as above. Percentage of monomeric species and Area (indicative of protein concentration) were determined for each molecule/freeze&thaw cycle and compared to a reference run (samples stored at 4° C.).

PetML119 showed good resistance to Freeze and Thaw, with only minor protein loss upon 3 cycles.

### Thermal & Chemical Stress:

50 μL aliquots of PetML119 at 5 mg/mL in PBS or PBS+100 mM DTT were prepared and incubated for 30′ at different temperatures (25° C., 37° C., 60° C., 70° C. and 95° C.).

40 μL of each condition were diluted with 10 μL of NuPAGE™ LDS Sample Buffer (NP0007). 10 uL were loaded into NuPAGE™ 10%, Bis-Tris, 1.0 mm, Mini Protein Gel, 12-well (NP0302BOX) and run at 120V for 45′. Gel was stained with InstantBlue® Coomassie Protein Stain (ISB1L) (ab119211) for 10′ and destained in ddH2O for an 1 hr. SDS-PAGE was then acquired using standard transilluminator equipment.

Results showed that PetML119 is very stable in both temperature and chemical stress, showing unfolding only when incubated at temperature higher than 70° C.

### 5. Protein A Binding Affinity Validation (FIG. 5)

Purified fusion proteins (PetML119 and PetML122) in PBS were concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to Protein A was assessed using Biacore 8K (Cytiva).

Briefly, Sensor Chip Protein A (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

Fusion protein dilutions were prepared diluting PetML119/122 from 1 uM to 4 nM (6 concentrations with 1:3 dilutions) in Running Buffer and kinetics was assessed using single cycle kinetics method (Biacore Assay Handbook, Cytiva). Kinetics and/or Affinity quantification have been performed using Biacore Insight following standard analyses methods.

The results show that protein A binding for PetML122 is slightly affected by YTE mutation introduced, a faster dissociation is observed compared to PetML119. This results in lower yield post protein A purification although the quality of protein is comparable.

### 6. FcRn Binding Affinity Validation (FIG. 6)

Purified fusion proteins (PetML119 and PetML122) in PBS were concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to Fc Neonatal Receptor was assessed using Biacore 8K (Cytiva).

Briefly, CM5 Sensor Chip (Cytiva) was docked into Biacore 8K, equilibrated for 30′ at RT and then Running Buffer (10 mM HEPES pH6 150 mM NaCl 3 mM EDTA and 0.005% Tween20) was applied to the SPR chip surface.

Canine and murine FcRn-B2M recombinant protein (Immunitrack, ITF12-ITF08) was diluted into 10 mM acetate buffer pH4.5 at 4 nM (1:2000 dilution from stock) and immobilised using standard amine coupling reaction

Fusion protein dilutions were prepared from 3 uM to 37 nM (5 concentrations with 1:3 dilutions) in Running Buffer and kinetics was assessed using multi-cycle kinetics method (120 sec association-300 sec dissociation). Kinetics and/or Affinity quantification have been performed using Biacore Insight following standard analyses methods.

A 10-fold increase in binding affinity to FcRn has been seen for PetML122, confirming the triple mutant YTE had a positive effect on binding to both canine and murine neonatal receptor. In particular, an extended dissociation rate was observed.

7. Human and Rat Nerve Growth Factor (h-rNGF) Binding Affinity Determination (FIG. 7)

Purified fusion protein (PetML119) in PBS was concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to human and rat NGF was assessed using Biacore 8K (Cytiva).

Briefly, Protein A Sensor Chip (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

PetML119 was diluted into running buffer at 6 nM concentration. These have been immobilised using 90 sec association at 10 uL/min as capturing step, followed by injection of running buffer to remove any unbound product.

Human and rat NGF (from Bio-Techne Ltd—556-NG/CF/256-GF-100/CF) was diluted in Running Buffer at 100 nM with 1:2 further dilutions down to 4.68 nM. Kinetics were assessed using multi-cycle kinetics with capture step method (30 sec association-120 sec dissociation) followed by regeneration step (0.1M Glycine pH2.2 contact time 60 sec FR 30 uL/min). Kinetics quantification have been performed using Biacore Insight following standard analyses methods.

Results showed subnanomolar KD for both human and rat NGF with PetML119.

### 8. Intact Mass Analyses

Purified fusion proteins (PetML119 and PetML122) in PBS were concentrated using centrifugal concentrators (Sartorious—VS02H22) to 1 mg/mL Protein concentration was assessed using UV absorbance at 280 nm with NanoDrop™ One (Thermo Scientific™).

LC-MS analyses have been performed on BioAccord using BioResolve RP mAb Polyphenyl Column, 450 Å, 2.7 μm, 2.1×50 mm and ACQUITY UPLC® I-Class Plus from WATERS.

Briefly, 3 μmol (diluted in LC-MS grade H2O) of each fusion protein were injected in RP column and a eluate peaks were analysed by MS. Intact mass analyses showed presence of multiple species corresponding to different degree of glycosylation, consistently on what observed by H-SCX.

The intact molecule appears to be a mixture of species. The observed mass is 96158-97836 Da. The mass of the base species (96158 Da) is consistent with a dimer of Fc fusion aa 20-241+4 glycans which is consistent with the total number of glycosylation sites expected in the molecule. Additional species are consistent with additional decoration of the glycans.

### 9. Unfolding and Oligomerisation Determination (FIG. 4)

Purified fusion proteins (PetML119) in PBS were concentrated using centrifugal concentrators (Sartorious—VS02H22) to 3 mg/mL Protein concentration was assessed using UV absorbance at 280 nm with NanoDrop™ One (Thermo Scientific™).

Tm and Tagg analyses have been performed on UnCle from Unchained labs using standard protocol.

Briefly, 10 μL of fusion protein have been used to determine unfolding and aggregation events during a temperature ramp (from 25° C. till 95° C.). As anticipated from previous results, PetML119 showed no aggregation up to 95° C. with Tm1 around 67° C.

### 10. In Vitro NGF Inhibition Assay (FIG. 8)

To assess biological activity of our p75 fusion protein, we used an NGF-dependent (cell line TF-1 cell line). The cells are completely dependent on interleukin 3 (IL-3) or granulocyte-macrophage colony-stimulating factor (GM-CSF) for long term growth. The cells do not respond to interleukin 5 (IL-5). TF-1 cells respond to a variety of other lymphokines and cytokines such as interleukin 1 (IL-1), interleukin 4 (IL-4), interleukin 6 (IL-6), interleukin 9 (IL-9), Interleukin 11 (IL-11), interleukin 13 (IL-13), stem cell factor (SCF), leukemia inhibitory factor (LIF) and nerve growth factor (NGF).

Proficient sequestration of NGF by our fusion protein will result in slower proliferation in comparison to control.

TF-1 cell line was bought from ATCC (CRL-2003) and kept in culture using standard aseptic methods using complete RPMI (10% FBS+2 mM I-Gln+10 ng/mL hNGF).

2 million TF-1 cells were labelled with 2.5 uM CFSE cell trace (Invitrogen—C34554) in 1 mL of RPMI only for 30′ at RT in the dark. Cells have been then washed 2× in complete RPMI media, counted again and seeded at 10000 cells/mL (1 mL total volume per well) in 24-well plate.

Dilution of PetML119/122 or controls (Bedinvetmab—anti-NGF IgG control, unrelated fusion protein and RPMI without NGF as negative control WHO Drug Information, Vol. 32, No. 4, 2018—pg 568) were diluted in 100 μL of RPMI media from 3 uM concentration till 91.25 nM. 100 μL of protein dilution was added to each well. Briefly, published Bedinvetmab aa sequence for both heavy chain and light chain have been used to generate codon optimised (for CHO expression) cDNA. Both HC and LC cDNA have been cloned in frame with standard peggy-bac plasmid. CHO have been then stably transfected as above.

Plates were analysed after 3 days. Briefly, 1 mL cell suspension were centrifuged 5′ at 300 g RT, washed 2 times with FACS buffer (PBS+3% FBS+3 mM EDTA) and finally resuspended in 100 μL of FACS buffer. Cells were acquired using CytoFLEX Flow Cytometer using following parameters (FSC: 20; SSC: 50; FITC: 1; threshold: 1313131). Cells were gated based on FITC fluorescence (more fluorescence less proliferation) and % of proliferation inhibition was calculated considering 100% inhibition TF-1 cells cultured in RPMI without NGF and 0% inhibition cells cultured with complete RPMI media. Graph pad was used to calculate IC50 values.

Both PetML119/122 and Bedinvetmab were able to inhibit TF-1 proliferation in a dose dependent manner.

TABLE 1

| | PetML119 | PetML122 | BedinvetmAb |
|---|---|---|---|
| IC50 (nM) | 15.25 | 5.91 | 1.645 |

11. MIA-Induced OA in Rats (Efficacy and pK)—FIGS. 9 to 12)

Efficacy and half-life for PetML119/122 were analysed using a rat model of OA. The detailed protocol is shown below. The model is shown in FIG. 9.

### Induction of Arthritis

Osteoarthritis was induced chemically by an intra-articular (I.A.) injection of 3 mg of monosodium-iodoacetate (MIA) (in 25 μL saline) into the right hind limb knee joint of the rat given under isoflurane anesthesia. While under anesthesia, ophthalmic ointment was applied to both eyes. The day of I.A. injection of MIA was counted as Day 0.

### Allocation to Treatment Groups

Baseline dynamic weight bearing (DWB) were measured for all rats. Body weight (BW) was also measured at the same time. Rats were anesthetized and MIA injected into the right Knee joint through the middle of the patellar tendon approximately perpendicular to the tibia (Intra-articular (I.A.)). Dose level for I.A. injection of MIA was selected based on previous literature report in rodents (Bove et al.: Weight bearing as a measure of disease progression and efficacy of anti-inflammatory compounds in a model of monosodium iodoacetate-induced osteoarthritis. Osteoarthritis Cartilage. 2003 November; 11(11):821-830). Animals showing a significant weight bearing difference between the MIA injected limb (right) and the healthy limb (left) were assigned to the study. Randomization was done based on both baseline DWB and BW (two variables randomization).

### Dynamic Weight Bearing (DWB) Evaluation

Dynamic Weight Bearing was evaluated using the BioSeb® automated DWB system according to the manufacturer's manual. A two-minute recording was done for each rat. Analysis of dynamic weight bearing data was done off-line using the BioSeb® software. The system automatically calculated the weight borne by each limb and the tail. Body weight was measured for each rat immediately before the DWB for each time of testing. DWB measurement was done at different time points as per schedule in Study Design. Total distance travelled was also noted during DWB data analysis.

Dosing with Test Items

Group 1-2 rats received intravenous (IV) injections of vehicle and Group 3-7 rats received IV injections with the test items at designated doses once on Day 3 as depicted in the table below. Group 8 rats received oral gavage dosing with dexamethasone once daily from Day 3-21 as depicted in the table below.

Monosodium-Iodoacetate (MIA)-Induced GA Rat Study with PETML119/122 Study design

TABLE 2

| | | TI Dose | TI Route, | TI Dosing | | |
|---|---|---|---|---|---|---|
| *Gr. | Group Treatment | level | Volume | schedule | DWB testing | N |
| 1 | Naive/Vehicle PBS | 0 | IV, 5 | Day 3, | Day 1 (baseline), | 10 |
| | | | mL/kg | QD | Days 3, 6, 14, 21 | |
| 2 | 3 mg MIA/Vehicle | 0 | IV, 5 | Day 3, | Day 1 (baseline), | 10 |
| | PBS | | mL/kg | QD | Days 3, 6, 14, 21 | |
| 3 | 3 mg MIA/Sample 1 | 0.5 mg/kg | IV, 5 | Day 3, | Day 1 (baseline), | 10 |
| | (PetML119), low | | mL/kg | QD | Days 3, 6, 14, 21 | |
| | dose | | | | | |
| 4 | 3 mg MIA/Sample 1 | 1.5 mg/kg | IV, 5 | Day 3, | Day 1 (baseline), | 10 |
| | (PetML119), mid | | mL/kg | QD | Days 3, 6, 14, 21 | |
| | dose | | | | | |
| 5 | 3 mg MIA/Sample 1 | 5 mg/kg | IV, 5 | Day 3, | Day 1 (baseline), | 10 |
| | (PetML119), high | | mL/kg | QD | Days 3, 6, 14, 21 | |
| | dose | | | | | |
| 6 | 3 mg MIA/Sample 2 | 5 mg/kg | IV, 5 | Day 3, | Day 1 (baseline), | 10 |
| | (PetML122) | | mL/kg | QD | Days 3, 6, 14, 21 | |
| 7 | 3 mg MIA/Sample 3 | 5 mg/kg | IV, 5 | Day 3, | Day 1 (baseline), | 10 |
| | (Bedinvetmab) | | mL/kg | QD | Days 3, 6, 14, 21 | |
| 8 | 3 mg MIA/ | 1 mg/kg from | PO, 10 | Days 3-21 | Day 1 (baseline), | 10 |
| | Dexamethasone | Day 1-7 and | ml/kg | QD | Days 3, 6, 14, 21 | |
| | | 0.3 mg/kg from | | | | |
| | | Day 8-21 | | | | |

*Animals from Groups 2 to 8 plus spares receive a single intra-articular (IA) injection with MIA (3 mg/25 μL saline) and the day of MIA injection is considered as Day 0 (in the right knee joint).
IV: Intravenous (tail vein);
PO: oral gavage;
QD—once daily

Treated animals were observed for any clinical signs during the study. DWB was analysed on Days 3, 6, 14 and 21. Joint diameter was measured using a caliper on the right knee joint (medio-laterally) on days 3, 6, 14 and 21.

Briefly, both PetML119 (at different doses) and 122 (single dose) showed good analgesic effect outcompeting anti-NGF benchmark (Bedinvetmab) (FIG. 10). They also showed a similar activity to daily administration of dexamethasone while not showing side effects of NSAIDs (body weight loss—FIG. 11).

PK bleeds from 5 rats per group were taken using standard procedures on alternative days.

### Serum pk Analysis (FIG. 12)

Sandwich ELISA to quantify serum levels of our fusion protein were set up as follows:
30 uL of 2 ug/mL of Capturing antibody (Mouse anti-canine p75 Ab->MAB367-SP (Novus Bio)) diluted in PBS+0.1M sodium bicarbonate were immobilised on half-area ELISA plates (MICROPLATE, 96 WELL, PS, HALF AREA, CLEAR, Item No.: 675061) overnight at 4° C.
Plates were washed 2× with 200 uL blocking solution (PBS+5% DNFM+0.2% Tween20) and blocking have been performed with 150 uL of blocking solution across all wells for 3 hrs at RT.
Sera from different timepoints/groups of rat study were diluted 100× in blocking solution (2 uL serum+198 uL blocking solution) and 30 uL were added to relevant wells. Standards from PetML119/122 were prepared diluting fusion protein into rat serum from 100 ug/mL till 1 ng/mL with 1:5 dilutions. Standards were then diluted 100× in blocking solution and 30 uL have been added to relevant wells.
Sera were incubated for 1 hr at RT with 450 rpm shaking.
Plates were washed 2× with 200 uL blocking solution; detection antibody (SA5-10309 (ThermoFisher)) have been diluted 1:40000 in blocking solution and 30 uL were added to each well and left 30′ at RT with 450 rpm shaking.
Plates were washed 2× with 200 uL blocking solution; developing HRP-conjugated antibody (A16035 (ThermoFisher)) were diluted 1:10000 in blocking solution and 30 uL were added to each well and left 30′ at RT with 450 rpm shaking.
Plates were washed 2× with 200 uL blocking solution then 2× with 200 μL of PBS+0.2% Tween20 and finally 50 uL of TMB (TMB Chromogen Solution (for ELISA)->002023) were added to each well. After 10′, when standard curve showed saturation in first two points, the reaction was stopped adding 50 uL of 1M Sulphuric acid.
All wells were read with CLARIOstar Plus (BMG LABTECH) using endpoint Absorbance at 650 nm and 450 nm. Values were imported in Graph Pad and one-phase decay fitting have been applied to estimate half-life of these.

PetML119, from different doses, showed similar half-life (around 70 hrs) while PetML122 showed an extended half-life (around 10.5 days), see FIG. 12.

12. Pharmacokinetic and Pharmacodynamic Study of PetML119 and PetML122 after OA Induction in Dogs

The safety profile, pharmacokinetic and pharmacodynamic parameters of PetML119 and PetML122 were evaluated after a single intravenous administration at 1.5 mg/kg in dogs after induction of OA (urate crystals model). The study was carried out similar to the protocol in Toutian et al, J. vet. Pharmacol. Therap. 24, 43-55, 2001.

This study was performed according to a 2-groups, 4-period, 4-treatments, and 3 sequences. Ten dogs were used.

Four treatments were tested:
A. Positive Control (Meloxicam) Per Os.
B. Negative Control (Vehicle) Intravenous infusion.
C. PetML119 (1.5 mg/kg) Intravenous infusion.
D. PetML122 (1.5 mg/kg) Intravenous infusion

TABLE 3

| Group No. | Period 1 | Period 2 | Period 3 | Period 4 |
|---|---|---|---|---|
| 1 | A | B | C | / |
| 2 | B | A | D | / |

A reversible urate crystal synovitis model was used to induce an experimental acute synovitis. A sodium urate crystal suspension was prepared at a concentration of 10 mg/mL. General anaesthesia was induced via an intra-articular injection of propofol. Alternate stifle joints were used in each period.

Vehicle and test item (PetML119/PetML122) treatment administrations (by intravenous delivery) were performed three days before acute synovitis induction (at Day-3). Meloxicam treatment administrations were performed right before acute synovitis induction (at T-0.5 h). Treatment administrations were designed to obtain maximum treatment effect at the time of maximal induced lameness (i.e. 2 h-3 h after acute synovitis induction).

TABLE 4

| Period | Study day | Event |
|---|---|---|
| Period 1 | Day −4 to day −1 | Efficacy assessments. Pre-induction (visual lameness score |
| | 13 to 16 Jan. 2022 | and measure of the peak vertical force) |
| | | Vehicle administration (3 days before synovitis |
| | | induction) group 2 |
| | | Overnight fasting (12 h at least) |
| | Day 0 | Animal weighing |
| | 17 Jan. 2022 | Meloxicam administration (30 mins before synovitis |
| | | induction) group 1 |
| | | Synovitis induction (T0) |
| | | Efficacy assessments: T1.5 h, T2.75 h post urate crystals |
| | | induction |
| | | Food distribution (after T2.75 h assessments) |
| | | Efficacy assessments: T4 h, T5.25 h, T6.5 h post urate crystals |
| | | induction |
| Period 2 | Day −4 to day −1 | Efficacy assessments. Pre-induction (visual lameness score |
| | 27 to 30 Jan. 2022 | and measure of the peak vertical force) |
| | | Vehicle administration (3 days before synovitis |
| | | induction) group 1 |
| | | Overnight fasting (12 h at least) |
| | Day 0 | Animal weighing |
| | 31 Jan. 2022 | Meloxicam administration (30 mins before synovitis |
| | | induction) group. |
| | | Synovitis induction (T0) |
| | | Efficacy assessments: T1.5 h, T2.75 h post urate crystals |
| | | induction |
| | | Food distribution (after T2.75 h assessments) |
| | | Efficacy assessments: T4 h, T5.25 h, T6.5 h post urate crystals |
| | | induction |
| Period 3 | Day −4 | Blood sampling for hematology biochemistry and plasma |
| | 10 Feb. 2022 | biomarkers T-24 h before test item administration |
| | | Efficacy assessments Pre-induction (visual lameness score |
| | | and measure of the peak vertical force) |
| | Day −3 | Predose blood sampling |
| | 11 Feb. 2022 | Test item administration (3 days before synovitis |
| | | induction) all groups |
| | | Overnight fasting (12 h at least) |
| | Day −2 | Blood sampling T24 h after test item administration. |
| | 12 Feb. 2022 | |
| | Day −1 | Blood sampling T48 h after test item administration. |
| | 13 Feb. 2022 | |
| | Day 0 | Blood sampling T72 h after test item administration. |
| | 14 Feb. 2022 | Animal weighing |
| | | Synovitis induction (T0) |
| | | Efficacy assessments: T1.5 h, T2.75 h post urate crystals |
| | | induction |
| | | Food distribution (after T2.75 h assessments) |
| | | Efficacy assessments: T4 h, T5.25 h, T6.5 h post urate crystals |
| | | induction |
| | Day 4 | Blood sampling T168 h after test item administration. |
| | 18 Feb. 2022 | |
| | Day 11 | Blood sampling T336 h after test item administration. |
| | 25 Feb. 2022 | Blood sampling for hematology biochemistry T336 h after test |
| | | item administration |
| | Day 18 | Blood sampling T504 h after test item administration. |
| | 4 Mar. 2022 | |
| Period 4 | Day −1 | Efficacy assessments Pre-induction (visual lameness score |
| | 10 Mar. 2022 | and measure of the peak vertical force) |
| | | Overnight fasting (12 h at least) |
| | Day 0 | Blood sampling T672 h after test item administration. |
| | 11 Mar. 2022 | Animal weighing |
| | | Synovitis induction (T0) |
| | | Efficacy assessments: T1.5 h, T2.75 h post urate crystals |
| | | induction |
| | | Food distribution (after T2.75 h assessments) |
| | | Efficacy assessments: T4 h T5.25 h, T6.5 h post urate crystals |
| | | induction |
| Animal | 28 Mar. 2022 | Blood sampling T1080 h (Day 45) after test item administration |
| Follow-up | 12 Apr. 2022 | Blood sampling T1440 h (Day 60) after test item administration |

### Safety

Each animal was observed at least once daily and no abnormal findings were reported.

Haematological parameters were collected and animals weighed at the time points indicated in Table 4. Blood samples were drawn from the jugular or cephalic vein at alternate sites.

Sandwich ELISA to identify potential anti-Drug Antibodies (ADA) in the serum generated after treatment with our fusion protein in-vivo were set up as follows:
30 uL of 2 ug/mL of either PetML119 or PetML122 (two plates) diluted in PBS+0.1M sodium bicarbonate were immobilised on half-area ELISA plates (MICROPLATE, 96 WELL, PS, HALF AREA, CLEAR, Item No.: 675061) overnight at 4° C.
Plates were washed 2× with 200 uL blocking solution (PBS+5% DNFM+0.2% Tween20) and blocking have been performed with 150 uL of blocking solution across all wells for 3 hrs at RT.
Sera from different timepoints/groups of rat/dog study were diluted 100× in blocking solution (2 uL serum+198 uL blocking solution) and 30 uL were added to relevant wells. Standards Standard anti PetML119/122 Ab (MAB: 367 from 1 ng/mL) have been prepared in 20 μL of rat/dog serum+Ab and then diluted with a 1 in 2 dilution. Standards were then diluted 100× in blocking solution and 30 uL have been added to relevant wells.
Sera were incubated for 1 hr at RT with 450 rpm shaking.
Plates were washed 2× with 200 uL blocking solution; detection antibody (SA5-10309 (ThermoFisher)) have been diluted 1:20000 in blocking solution and 30 uL were added to each well and left 30′ at RT with 450 rpm shaking.
Plates were washed 2× with 200 uL blocking solution; developing HRP-conjugated antibody (A16035 (ThermoFisher)) were diluted 1:10000 in blocking solution and 30 uL were added to each well and left 30′ at RT with 450 rpm shaking.
Plates were washed 2× with 200 uL blocking solution then 2× with 200 μL of PBS+0.2% Tween20 and finally 50 uL of TMB (TMB Chromogen Solution (for ELISA)->002023) were added to each well. After 10′, when standard curve showed saturation in first two points, the reaction was stopped adding 50 uL of 1M Sulphuric acid.
All wells were read with CLARIOstar Plus (BMG LABTECH) using endpoint Absorbance at 650 nm and 450 nm. Values were imported in Graph Pad and one-phase decay fitting have been applied to estimate half-life of these.

### Results

Bodyweight (FIG. 13a) and haematological parameters were unaltered after PetML119/PetML122 administration.

For ADA analysis (FIG. 13b), both sera from animals treated with PetML119/PetML122 showed basal signal comparable with the pre-dose control, indicating no anti-drug antibodies had been generated during the experimental time frame.

### Efficacy

To assess the anti-inflammatory and analgesic efficacy of the test item, two parameters were investigated: visual lameness score (VLS, determined using a scoring system) and the vertical force (VF, expressed in Newtons) applied to the ground for the hind limb measured with a force plate. The force plate was connected to a computer equipped with a digital analogical acquisition card and a signal processing software. The force plate (made with 8 scales) was inserted in a path 50 cm wide and about 5 m long along which the dogs were trained to walk on a lead at a constant and similar speed. Based on the maximum treatment effect and maximal induced lameness, the efficacy assessment was performed pre-induction and 1.5 h, 2.75 h, 4 h, 5.25 h and 6.5 h (±10 min per timepoint) post-induction.

### Lameness Ratio

The VF values were obtained as the limb is placed on the force plate. The dogs passed at least 5 times in the path of the force plate in order to obtain 3 interpretable values for the hind limb, to calculate the mean force of the limb. The ratio between the force applied after treatment and the force applied on Predose (reference force) of the same hind limb was referred to as the ‘lameness ratio.’ This parameter describes the force applied by the induced limb in relation to the force applied in the absence of inflammation. If severe lameness with no weight bearing is observed during walking phase, the vertical force was not measured and the lameness ratio was considered equal to zero.

### Visual Lameness Score

The dogs were scored for lameness whilst standing and whilst walking (for approximately one minute before scoring), according to the semi-quantitative scales detailed as follows:

TABLE 5

| Observation whilst standing | Score |
|---|---|
| Full weight bearing (touching of all | 0 |
| digital pads on the ground) | |
| Partial weight bearing (touching of just | 1 |
| a part of digital pads on the ground) | |
| No weight bearing to toe touching | 2 |

TABLE 6

| Observation whilst walking | Score |
|---|---|
| Full weight bearing, no lameness | 0 |
| Slight lameness with partial weight bearing: | 1 |
| lameness barely perceptible | |
| Moderate lameness with partial weight bearing: | 2 |
| the animal rests the limb on the ground slightly | |
| Severe lameness with no weight bearing: | 3 |
| the animal uses its limb but does not put | |
| its weight on the limb and/or avoid | |
| putting the limb on the ground | |

The combined lameness visual score was considered as the sum of the standing and walking phase scores.

### Results

After treatment, both PetML119 and PetML122 exhibit improved efficacy to the gold standard for acute pain, Meloxicam (FIG. 14). Furthermore, PetML119 and PetML122 have the additional benefit of only requiring a single dose for extended pain relief. In contrast, Meloxicam or other existing treatments require daily administration which reduces compliance and increases stress to the animal.

### Serum pk Analysis

Blood samples were collected before and during period 3 at the following target time points: After intravenous test item administration at T24 h, T48 h, T72 h, (pre-induction Period 3) and T168 h, T336 h, T504 h (pre-induction Period 4) and T672 h post dosing. Additional blood samples were collected at 45 and 60 days post dosing.

Sandwich ELISA to measure pK values are as described in Example 11 above.

### Results

PetML122 showed an extended half-life (around 19 days) in comparison to the half-life of PetML119 (around 4 days), see FIG. 15.

### Sequences

canine p75NTR protein
SEQ ID NO. 1
KEACPTGLYTHSGECCKACNLGEGVAQPCGANQTVCEPCLDSVTFSDVVSATEPCKPCTE
CVGLQSMSAPCVEADDAVCRCAYGYYQDETTGRCEACRVCEAGSGLVFSCQDRQNTVCEE
CPDGTYSDEANHVDPCLPCTVCEDTERQLRECTRWADAECEEIPGRWITRSTPSEDSDST
APSTEEPELPPDQEIIASTMADVVTTVMGSSQPVVTRGTADNLIPVYCSILAAVVVGLVA
YIAFKRWNSCKQNKQGANSRPVNQTPPPEGEKLHSDSGISVDSQSLHDQQPHTQTAAGQA
LKGDGGLYSSLPPAKREEVEKLLNGSAGDTWRHLAGELGYQPEHIDSFTHEACPARALLA
SWAAQDSATLDALLAALRRIQRADIVESLCSESTATSPV
ECD is underlined
canine p75NTR nucleic acid sequence
SEQ ID NO. 2
ATGGACGGGCCGCGCCTGCTGCTGCTGCTGCTGCTGCTCCTGGGGGTGTCCCTTGGAGGTGCC
AAGGAGGCATGTCCCACTGGCCTGTACACCCACAGCGGCGAGTGCTGCAAAGCCTGCAATCTGG
GTGAGGGGGTGGCCCAGCCTTGCGGAGCCAACCAGACCGTGTGTGAGCCCTGCCTGGACAGCG
TGACCTTCTCGGACGTGGTGAGCGCCACCGAGCCGTGCAAGCCGTGCACCGAGTGCGTGGGGC
TGCAGAGCATGTCGGCGCCGTGCGTGGAGGCGGACGACGCCGTGTGCCGCTGCGCCTACGGC
TACTACCAGGACGAGACGACGGGCCGCTGCGAGGCGTGCCGCGTGTGCGAGGCGGGCTCGGG
GCTCGTGTTCTCGTGCCAGGACAGGCAGAACACCGTGTGCGAGGAGTGTCCCGACGGCACGTA
CTCCGACGAGGCCAACCACGTGGACCCGTGCCTGCCCTGCACCGTGTGCGAGGACACCGAGCG
CCAGCTGCGCGAGTGCACGCGCTGGGCCGACGCCGAGTGCGAGGAGATCCCTGGCCGTTGGA
TTACCCGGTCCACACCCTCAGAGGACTCGGACAGCACCGCCCCCAGCACAGAGGAGCCAGAGC
TACCTCCAGATCAAGAAATCATAGCCAGCACCATGGCAGATGTGGTGACCACAGTGATGGGCAG
CTCTCAGCCTGTAGTGACCCGAGGAACCGCTGACAACCTCATCCCTGTCTACTGCTCCATCCTGG
CCGCCGTGGTTGTGGGCTTAGTGGCCTACATTGCCTTCAAGAGGTGGAACAGCTGCAAGCAGAA
CAAGCAAGGAGCCAACAGCCGGCCCGTGAACCAGACGCCTCCGCCGGAGGGAGAAAAGCTCCA
CAGTGACAGTGGCATCTCTGTGGACAGCCAGAGCCTGCATGACCAGCAGCCCCACACACAGACG
GCCGCAGGCCAGGCCCTCAAGGGGGATGGAGGTCTCTACAGCAGCCTGCCACCAGCCAAGCGG
GAGGAGGTGGAGAAGCTGCTCAATGGCTCTGCGGGGGACACCTGGCGGCACCTGGCAGGTGA
GCTGGGCTACCAGCCTGAGCACATAGACTCCTTCACCCACGAGGCCTGCCCAGCCCGAGCCCT
GCTTGCCAGCTGGGCCGCCCAGGACAGCGCGACGCTCGACGCCCTCCTGGCTGCTCTGCGCCG
CATCCAGCGAGCCGACATCGTGGAGAGCCTGTGTAGCGAGTCCACGGCCACGTCTCCAGTGTG
A
Leader sequence is underlined
feline p75NTR protein
SEQ ID NO. 3
KEACPTGLFTHSGECCKACNLGEGVAQPCGANQTVCEPCLDSVTFSDVVSATEPCKPCTE
CVGLQSMSAPCVEADDAVCRCAYGYYQDETTGRCEACRVCEAGSGLVFSCQDRQNTVCEE
CPDGTYSDEANHVDPCLPCTVCEDTERQLRECTRWADAECEEIPGRWITRSTPSEGSDST
APSTEEPEVPPEQDLIASTVADVVTTVMGSSQPVVTRGTADNLIPVYCSILAAVVVGLVA
YIAFKRWNSCKQDKQGANSRPVNQTPPPEGEKLHSDSGISVDSQSLHDQQSHTQTAAGQA
LKGDGGLYSSLPSAKREEVEKLLNGSAGDTWRHLAGELGYQPEHIDSFTREACPARALLA
SWAAQDSATLDALLAALRRIQRADIVESLCSESTATSPV
ECD is underlined
feline p75NTR nucleic acid sequence
SEQ ID NO. 4
ATGGACGGGCCGCGCCCGCTGCTGCTGCTGTTGCCGCTGCTCCTGGGGGTGTCCCTTGGAGGT
GCCAAGGAGGCATGTCCCACGGGCCTGTTCACCCACAGCGGCGAGTGCTGTAAAGCCTGCAAC
CTGGGAGAGGGCGTAGCCCAGCCTTGCGGAGCCAACCAGACCGTGTGTGAGCCCTGCCTGGAC
AGCGTGACCTTCTCGGACGTGGTGAGCGCCACGGAGCCGTGCAAGCCGTGCACCGAGTGCGTG
GGCCTGCAGAGCATGTCGGCGCCGTGCGTGGAGGCCGACGACGCCGTGTGTCGCTGCGCCTA
CGGCTACTACCAGGACGAGACGACGGGCCGCTGCGAGGCGTGCCGCGTGTGCGAGGCGGGCT
CCGGCCTGGTGTTCTCGTGCCAGGACCGGCAGAATACCGTGTGCGAGGAGTGTCCCGACGGCA
CGTACTCGGACGAGGCCAACCACGTGGACCCGTGCCTGCCCTGCACCGTGTGCGAGGACACCG
AGCGCCAGCTGCGCGAGTGCACGCGCTGGGCCGACGCCGAGTGCGAGGAGATCCCTGGCCGT
TGGATTACTCGGTCTACACCTTCGGAGGGCTCGGACAGCACCGCCCCCAGCACGGAGGAGCCA
GAGGTACCTCCAGAGCAAGACCTCATAGCCAGCACGGTGGCAGATGTGGTGACCACAGTGATGG
GCAGCTCTCAGCCCGTAGTGACCCGAGGCACCGCCGACAACCTCATCCCTGTCTATTGTTCCAT
CCTGGCCGCTGTGGTTGTGGGCCTGGTGGCCTACATTGCCTTCAAGAGGTGGAACAGCTGCAAA
CAGGACAAGCAAGGCGCCAACAGCCGGCCCGTGAACCAGACGCCCCCGCCCGAGGGAGAAAA
GCTCCACAGTGACAGTGGCATCTCTGTGGACAGCCAGAGCCTGCATGACCAGCAGTCCCACACG
CAGACGGCCGCCGGCCAGGCCCTCAAGGGGGACGGAGGTCTCTACAGCAGCCTGCCGTCAGC
CAAGCGGGAGGAGGTAGAGAAACTGCTCAACGGCTCTGCGGGGGACACGTGGCGGCACCTGG
CGGGCGAGCTGGGCTACCAGCCTGAGCACATAGACTCCTTCACCCGCGAGGCCTGCCCAGCCC
GGGCCCTGCTCGCCAGCTGGGCCGCCCAGGACAGCGCGACGCTCGACGCCCTCCTGGCCGCC
CTGCGCCGCATCCAGCGGGCCGACATCGTCGAGAGCCTGTGCAGCGAGTCCACGGCCACGTCC
CCGGTGTGA
Leader sequence is underlined
equine p75NTR protein
SEQ ID NO. 5
KEVCPTDLYTHSGECCKACNLGEGVAQPCGANQTVCEPCLDSVTFSDVVSATEPCKPCTE
CVGLQSMSAPCVEADDAVCRCAYGYYQDETTGRCEACQVCEAGSGLVFSCQDKQNTVCEE
CPDGTYSDEANHVDPCLPCTVCEDTERQLRECTRWADAECEEIPSRWITRATPPEGSDST
APSTQEPEGPPEKDLVASTVADVVTTVMGSSQPVVTRGTTDNLIPVYCSILAAVVVGLVA
YIAFKRWNSCKQNKQGANSRPVNQTPPPEGEKLHSDSGISVDSQSLHDQQPHTQTAAGQA
LKGDGGLYSSLPLAKREEVEKLLNGSAGDTWRHLAGLVGQGLLRLELVSVFQGPAHGGML
PPATPSLQAPVWLGPEGCSEKWDQRGNAARRAGLRVWPMEGLSQV
ECD is underlined
equine p75NTR nucleic acid sequence
SEQ ID NO. 6
ATGAGGGCAGGTGCCGCCGACTGCGCCATGGACGGACCGCGCCTTCTGCTGCTGCTTCTGCTC
TTGGGGGTGTGCCTGCTGGGAGGTGCCAAGGAGGTGTGCCCCACAGACCTGTACACCCACAGC
GGCGAGTGCTGCAAAGCCTGCAACCTGGGCGAGGGTGTGGCCCAGCCTTGCGGAGCCAACCAG
ACTGTGTGTGAACCCTGCCTGGACAGCGTGACGTTCTCGGACGTGGTGAGCGCCACAGAGCCAT
GTAAGCCGTGCACCGAGTGCGTGGGCCTGCAGAGCATGTCGGCGCCATGCGTGGAGGCCGAC
GACGCGGTGTGCCGCTGCGCCTATGGCTACTACCAGGACGAGACGACGGGCCGCTGCGAGGC
GTGCCAGGTGTGCGAGGCGGGCTCGGGCCTCGTGTTCTCGTGCCAGGACAAGCAGAACACCGT
GTGCGAGGAATGCCCCGACGGCACGTACTCCGACGAGGCCAACCACGTGGACCCGTGCCTGCC
CTGCACCGTGTGCGAGGACACCGAGCGCCAGCTGCGAGAGTGCACGCGCTGGGCCGACGCCG
AGTGCGAGGAGATCCCCAGCCGTTGGATTACACGGGCCACGCCGCCGGAGGGCTCAGACAGCA
CTGCCCCCAGCACCCAGGAGCCCGAGGGACCTCCAGAGAAAGACCTTGTAGCCAGCACGGTGG
CGGATGTGGTGACCACAGTGATGGGCAGCTCTCAGCCCGTGGTGACCCGAGGCACCACGGACA
ACCTCATCCCCGTCTATTGCTCCATCCTGGCCGCTGTGGTTGTGGGCCTTGTGGCCTACATCGC
CTTCAAGAGGTGGAACAGCTGCAAGCAGAACAAGCAAGGAGCCAACAGCCGACCCGTGAACCA
GACACCACCACCCGAGGGAGAAAAACTCCACAGCGACAGCGGCATCTCTGTGGACAGCCAGAG
CCTGCATGACCAGCAGCCTCACACACAGACAGCCGCAGGCCAGGCCCTCAAGGGAGATGGAGG
CCTCTACAGCAGCCTGCCACTGGCCAAGAGGGAAGAGGTGGAGAAGCTACTCAATGGCTCCGCA
GGGGACACCTGGCGGCACCTGGCGGGTGAGCTGGGCTACCAGCCCGAGCACATAGACTCCTTC
ACCCACGAGGCCTGCCCCGTCCGCGCCCTGCTTGCCAGCTGGGCCGCCCAGGACAGTGCGACA
TTCGATGCCCTCCTGACCGCCCTGCGCCGCATCCAGCGAGCCGACATTGTCGAGAGCCTGTGCA
GCGAGTCCACCGCCACATCCCCGGTGTGA
Leader sequence is underlined
canine p75NTR protein ECD
SEQ ID NO. 7
KEACPTGLYTHSGECCKACNLGEGVAQPCGANQTVCEPCLDSVTFSDVVSATEPCKPCTECVGLQS
MSAPCVEADDAVCRCAYGYYQDETTGRCEACRVCEAGSGLVFSCQDRQNTVCEECPDGTYSDEAN
HVDPCLPCTVCEDTERQLRECTRWADAECEEIPGRWITRSTPSEDSDSTAPSTEEPELPPDQEIIAST
MADVVTTVMGSSQPVVTRGTADN
The wt ECD region includes the stalk region (underlined)
and alpha and gamma secretase cleavage 3′ of the stalk
region (in bold)
canine p75NTR ECD nucleic acid sequence
SEQ ID NO. 8
AAGGAGGCATGTCCCACTGGCCTGTACACCCACAGCGGCGAGTGCTGCAAAGCCTGCAATCTGG
GTGAGGGGGTGGCCCAGCCTTGCGGAGCCAACCAGACCGTGTGTGAGCCCTGCCTGGACAGCG
TGACCTTCTCGGACGTGGTGAGCGCCACCGAGCCGTGCAAGCCGTGCACCGAGTGCGTGGGGC
TGCAGAGCATGTCGGCGCCGTGCGTGGAGGCGGACGACGCCGTGTGCCGCTGCGCCTACGGC
TACTACCAGGACGAGACGACGGGCCGCTGCGAGGCGTGCCGCGTGTGCGAGGCGGGCTCGGG
GCTCGTGTTCTCGTGCCAGGACAGGCAGAACACCGTGTGCGAGGAGTGTCCCGACGGCACGTA
CTCCGACGAGGCCAACCACGTGGACCCGTGCCTGCCCTGCACCGTGTGCGAGGACACCGAGCG
CCAGCTGCGCGAGTGCACGCGCTGGGCCGACGCCGAGTGCGAGGAGATCCCTGGCCGTTGGA
TTACCCGGTCCACACCCTCAGAGGACTCGGACAGCACCGCCCCCAGCACAGAGGAGCCAGAGC
TACCTCCAGATCAAGAAATCATAGCCAGCACCATGGCAGATGTGGTGACCACAGTGATGGGCAG
CTCTCAGCCTGTAGTGACCCGAGGAACCGCTGACAAC
canine ECD of p75NTR stalk region protein
SEQ ID NO. 9
WITRSTPSEDSDSTAPSTEEPELPPDQEIIASTMADVVTTVM
canine ECD of p75NTR stalk region nucleic acid sequence
SEQ ID NO. 10
TGGATTACCCGGTCCACACCCTCAGAGGACTCGGACAGCACCGCCCCCAGCACAGAGGAGCCA
GAGCTACCTCCAGATCAAGAAATCATAGCCAGCACCATGGCAGATGTGGTGACCACAGTGATG
canine p75NTR ECD-canine IgGB wt Fc protein fusion
SEQ ID NO. 11
MEWSWVFLFFLSVTTGVHSKEACPTGLYTHSGECCKACNLGEGVAQPCGANQTVCEPCLDSVTFSD
VVSATEPCKPCTECVGLQSMSAPCVEADDAVCRCAYGYYQDETTGRCEACRVCEAGSGLVFSCQD
RQNTVCEECPDGTYSDEANHVDPCLPCTVCEDTERQLRECTRWADAECEEIPGGGGRENGRVPRP
PDCPKCPAPEMLGGPSVFIFPPKPKDTLLIARTPEVTCVVVDLDPEDPEVQISWFVDGKQMQTAKTQP
REEQFNGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWQRGDTFIC
AVMHEALHNHYTQKSLSHSPGK
Signal peptide
Canine p75-ECD
Linker GGGG
Canine Fc-B wt
canine p75NTR ECD-canine IgGB wt Fc nucleic acid sequence
SEQ ID NO. 12
ATGGAATGGTCCTGGGTGTTCCTGTTCTTCCTGTCCGTGACCACCGGCGTGCACTCCAAAGAGG
CTTGTCCTACCGGCCTGTACACCCACTCTGGCGAGTGTTGCAAGGCCTGTAATCTCGGCGAAGG
CGTGGCACAACCTTGTGGCGCTAATCAGACAGTGTGCGAGCCTTGCCTGGACTCCGTGACCTTC
TCTGATGTGGTGTCTGCCACCGAGCCATGCAAGCCTTGTACCGAGTGTGTGGGCCTGCAGTCCA
TGTCTGCCCCTTGTGTGGAAGCCGACGACGCCGTGTGTAGATGTGCCTACGGCTACTACCAGGA
CGAGACAACCGGAAGATGCGAGGCCTGCAGAGTGTGTGAAGCTGGCTCTGGACTGGTGTTCTCC
TGCCAAGACAGACAGAACACCGTGTGCGAGGAATGCCCTGACGGCACCTACTCTGATGAGGCCA
ATCACGTGGACCCCTGCCTGCCTTGTACTGTGTGCGAAGATACCGAGCGGCAGCTGCGCGAGTG
TACCAGATGGGCTGATGCCGAGTGCGAAGAGATCCCTGGAGGTGGCGGACGCGAGAATGGCAG
AGTGCCTAGACCTCCTGACTGCCCTAAGTGCCCTGCTCCTGAAATGCTCGGCGGACCCTCCGTG
TTCATCTTCCCACCTAAGCCTAAGGACACCCTGCTGATCGCTCGGACCCCTGAAGTGACATGCGT
GGTGGTGGATCTGGACCCCGAGGATCCTGAGGTGCAGATCAGTTGGTTCGTGGACGGCAAGCA
GATGCAGACCGCTAAGACCCAGCCTAGAGAGGAACAGTTCAACGGCACCTACAGAGTGGTGTCT
GTGCTGCCTATCGGCCACCAGGATTGGCTGAAGGGCAAGCAGTTTACCTGCAAAGTGAACAACA
AGGCCCTGCCTTCTCCAATCGAGCGGACCATCTCTAAGGCCAGAGGCCAGGCTCATCAGCCTTC
CGTGTATGTCCTGCCACCTAGCCGCGAGGAACTGTCCAAGAACACCGTGTCTCTGACCTGCCTG
ATCAAGGACTTCTTCCCTCCTGACATCGACGTGGAATGGCAGTCCAACGGCCAGCAAGAGCCCG
AGTCTAAGTACCGGACAACCCCTCCACAGCTGGACGAGGACGGCTCCTACTTCCTGTACTCCAA
GCTGTCCGTGGACAAGTCTCGGTGGCAGAGAGGCGACACCTTCATCTGTGCTGTGATGCACGAG
GCCCTGCACAACCACTACACCCAGAAGTCCCTGTCTCACTCCCCTGGCAAGTGA
Leader sequence is underlined
canine p75NTR ECD-canine Fc YTE protein fusion
SEQ ID NO. 13
MEWSWVFLFFLSVTTGVHSKEACPTGLYTHSGECCKACNLGEGVAQPCGANQTVCEPCL
DSVTFSDVVSATEPCKPCTECVGLQSMSAPCVEADDAVCRCAYGYYQDETTGRCEACRVC
EAGSGLVFSCQDRQNTVCEECPDGTYSDEANHVDPCLPCTVCEDTERQLRECTRWADAE
CEEIPGGGGRENGRVPRPPDCPKCPAPEMLGGPSVFIFPPKPKDTLYITREPEVTCVVVDL
DPEDPEVQISWFVDGKQMQTAKTQPREEQFNGTYRVVSVLPIGHQDWLKGKQFTCKVNNK
ALPSPIERTISKARGQAHQPSVYVLPPSREELSKNTVSLTCLIKDFFPPDIDVEWQSNGQQE
PESKYRTTPPQLDEDGSYFLYSKLSVDKSRWQRGDTFICAVMHEALHNHYTQKSLSHSPGK
Signal peptide
Canine p75-ECD
Linker GGGG
Canine Fc-B-YTE
canine p75NTR ECD-Fc YTE nucleic acid sequence
SEQ ID NO. 14
ATGGAATGGTCCTGGGTGTTCCTGTTCTTCCTGTCCGTGACCACCGGCGTGCACTCCAAAGAGG
CTTGTCCTACCGGCCTGTACACCCACTCTGGCGAGTGTTGCAAGGCCTGTAATCTCGGCGAAGG
CGTGGCACAACCTTGTGGCGCTAATCAGACAGTGTGCGAGCCTTGCCTGGACTCCGTGACCTTC
TCTGATGTGGTGTCTGCCACCGAGCCATGCAAGCCTTGTACCGAGTGTGTGGGCCTGCAGTCCA
TGTCTGCCCCTTGTGTGGAAGCCGACGACGCCGTGTGTAGATGTGCCTACGGCTACTACCAGGA
CGAGACAACCGGAAGATGCGAGGCCTGCAGAGTGTGTGAAGCTGGCTCTGGACTGGTGTTCTCC
TGCCAAGACAGACAGAACACCGTGTGCGAGGAATGCCCTGACGGCACCTACTCTGATGAGGCCA
ATCACGTGGACCCCTGCCTGCCTTGTACTGTGTGCGAAGATACCGAGCGGCAGCTGCGCGAGTG
TACCAGATGGGCTGATGCCGAGTGCGAAGAGATCCCTGGAGGTGGCGGACGCGAGAATGGCAG
AGTGCCTAGACCTCCTGACTGCCCTAAGTGCCCTGCTCCTGAAATGCTCGGCGGACCCTCCGTG
TTCATCTTCCCACCTAAGCCTAAGGACACCCTGTATATCACTCGGGAACCTGAAGTGACATGCGT
GGTGGTGGATCTGGACCCCGAGGATCCTGAGGTGCAGATCAGTTGGTTCGTGGACGGCAAGCA
GATGCAGACCGCTAAGACCCAGCCTAGAGAGGAACAGTTCAACGGCACCTACAGAGTGGTGTCT
GTGCTGCCTATCGGCCACCAGGATTGGCTGAAGGGCAAGCAGTTTACCTGCAAAGTGAACAACA
AGGCCCTGCCTTCTCCAATCGAGCGGACCATCTCTAAGGCCAGAGGCCAGGCTCATCAGCCTTC
CGTGTATGTCCTGCCACCTAGCCGCGAGGAACTGTCCAAGAACACCGTGTCTCTGACCTGCCTG
ATCAAGGACTTCTTCCCTCCTGACATCGACGTGGAATGGCAGTCCAACGGCCAGCAAGAGCCCG
AGTCTAAGTACCGGACAACCCCTCCACAGCTGGACGAGGACGGCTCCTACTTCCTGTACTCCAA
GCTGTCCGTGGACAAGTCTCGGTGGCAGAGAGGCGACACCTTCATCTGTGCTGTGATGCACGAG
GCCCTGCACAACCACTACACCCAGAAGTCCCTGTCTCACTCCCCTGGCAAGTGA
Leader sequence is underlined
IgG-A
SEQ ID NO. 15
MEFVLGWVFLVAILQGVQGEVQLVESGGDLVKPAGSLRLSCVASGFTFSNNAMNWVRQAPGKGLQ
WVAGINSGGSTASADAVKGRFTISRDNAKNTVYLQMNSLTAEDTAVYYCAKVIGNWIATSDLDYWGQ
GTLVIVSSASTTAPSVFPLAPSCGSTSGSTVALACLVSGYFPEPVTVSWNSGSLTSGVHTFPSVLQSS
GLYSLSSMVTVPSSRWPSETFTCNVVHPASNTKVDKPVFNECRCTDTPPCPVPEPLGGPSVLIFPPK
PKDILRITRTPEVTCVVLDLGREDPEVQISWFVDGKEVHTAKTQSREQQFNGTYRVVSVLPIEHQDWL
TGKEFKCRVNHIDLPSPIERTISKARGRAHKPSVYVLPPSPKELSSSDTVSITCLIKDFYPPDIDVEWQS
NGQQEPERKHRMTPPQLDEDGSYFLYSKLSVDKSRWQQGDPFTCAVMHETLQNHYTDLSLSHSPG
K
IgG-B
SEQ ID NO. 16
MEFVLGWVFLVAILQGVQGEVQLVESGGDLVKPAGSLRLSCVASGFTFSNNAMNWVRQAPGKGLQ
WVAGINSGGSTASADAVKGRFTISRDNAKNTVYLQMNSLTAEDTAVYYCAKVIGNWIATSDLDYWGQ
GTLVIVSSASTTAPSVFPLAPSCGSTSGSTVALACLVSGYFPEPVTVSWNSGSLTSGVHTFPSVLQSS
GLYSLSSMVTVPSSRWPSETFTCNVAHPASKTKVDKPVPKRENGRVPRPPDCPKCPAPEMLGGPSV
FIFPPKPKDTLLIARTPEVTCVVVDLDPEDPEVQISWFVDGKQMQTAKTQPREEQFNGTYRVVSVLPIG
HQDWLKGKQFTCKVNNKALPSPIERTISKARGQAHQPSVYVLPPSREELSKNTVSLTCLIKDFFPPDID
VEWQSNGQQEPESKYRTTPPQLDEDGSYFLYSKLSVDKSRWQRGDTFICAVMHEALHNHYTQKSLS
HSPGK
IgG-C
SEQ ID NO. 17
MEFVLGWVFLVAILQGVQGEVQLVESGGDLVKPAGSLRLSCVASGFTFSNNAMNWVRQAPGKGLQ
WVAGINSGGSTASADAVKGRFTISRDNAKNTVYLQMNSLTAEDTAVYYCAKVIGNWIATSDLDYWGQ
GTLVIVSSASTTAPSVFPLAPSCGSQSGSTVALACLVSGYIPEPVTVSWNSGSLTSGVHTFPSILQSSG
LYSLSSMVTVPSSRWPSETFTCNVAHPATNTKVDKPVVKECECKCNCNNCPCPGCGLLGGPSVFIFP
PKPKDILVTARTPTVTCVVVDLDPENPEVQISWFVDSKQVQTANTQPREEQSNGTYRVVSVLPIGHQD
WLSGKQFKCKVNNKALPSPIEEIISKTPGQAHQPNVYVLPPSRDEMSKNTVTLTCLVKDFFPPEIDVE
WQSNGQQEPESKYRMTPPQLDEDGSYFLYSKLSVDKSRWQRGDTFICAVMHEALHNHYTQKSLSH
SPGK
IgG-D
SEQ ID NO. 18
MEFVLGWVFLVAILQGVQGEVQLVESGGDLVKPAGSLRLSCVASGFTFSNNAMNWVRQAPGKGLQ
WVAGINSGGSTASADAVKGRFTISRDNAKNTVYLQMNSLTAEDTAVYYCAKVIGNWIATSDLDYWGQ
GTLVIVSSASSTAPSVFPLAPSCGSTSGSTVALACLVSGYFPEPVTVSWNSGSLTSGVHTFPSVLKSS
GLYSLSSMVTVPSSRLPSETFTCNVVHPATNTKVDKPVPKESTCKCISPCPVPESLGGPSVFIFPPKPK
DILRITRTPEVTCVVLDLGREDPEVQISWFVDGKEVHTAKTQPREQQFNSTYRVVSVLPIEHQDWLTG
KEFKCRVNHIGLPSPIERTISKARGQAHQPGVYVLPPSPKELSSSDTVTLTCLIKDFFPPEIDVEWQSN
GQPEPESKYHTTAPQLDEDGSYFLYSKLSVDKSRWQQGDPFTCAVMHEALQNHYTDLSLSHSPGK
DOGA constant region
SEQ ID NO. 19
FNECRCTDTPPCPVPEPLGGPSVLIFPPKPKDILRITRTPEVTCVVLDLGREDPEVQISWFVDGKEVHT
AKTQSREQQFNGTYRVVSVLPIEHQDWLTGKEFKCRVNHIDLPSPIERTISKARGRAHKPSVYVLPPS
PKELSSSDTVSITCLIKDFYPPDIDVEWQSNGQQEPERKHRMTPPQLDEDGSYFLYSKLSVDKSRWQ
QGDPFTCAVMHETLQNHYTDLSLSHSPGK
DOGB constant region
SEQ ID NO. 20
RENGRVPRPPDCPKCPAPEMLGGPSVFIFPPKPKDTLLIARTPEVTCVVVDLDPEDPEVQISWFVDGK
QMQTAKTQPREEQFNGTYRVVSVLPIGHQDWLKGKQFTCKVNNKALPSPIERTISKARGQAHQPSVY
VLPPSREELSKNTVSLTCLIKDFFPPDIDVEWQSNGQQEPESKYRTTPPQLDEDGSYFLYSKLSVDKS
RWQRGDTFICAVMHEALHNHYTQKSLSHSPGK
DOGB-YTE constant region
SEQ ID NO. 21
RENGRVPRPPDCPKCPAPEMLGGPSVFIFPPKPKDTLYITREPEVTCVVVDLDPEDPEVQISWFVDGK
QMQTAKTQPREEQFNGTYRVVSVLPIGHQDWLKGKQFTCKVNNKALPSPIERTISKARGQAHQPSVY
VLPPSREELSKNTVSLTCLIKDFFPPDIDVEWQSNGQQEPESKYRTTPPQLDEDGSYFLYSKLSVDKS
RWQRGDTFICAVMHEALHNHYTQKSLSHSPGK
DOGC constant region
SEQ ID NO. 22
ECECKCNCNNCPCPGCGLLGGPSVFIFPPKPKDILVTARTPTVTCVVVDLDPENPEVQISWFVDSKQV
QTANTQPREEQSNGTYRVVSVLPIGHQDWLSGKQFKCKVNNKALPSPIEEIISKTPGQAHQPNVYVLP
PSRDEMSKNTVTLTCLVKDFFPPEIDVEWQSNGQQEPESKYRMTPPQLDEDGSYFLYSKLSVDKSR
WQRGDTFICAVMHEALHNHYTQKSLSHSPGK
DOGD constant region
SEQ ID NO. 23
ESTCKCISPCPVPESLGGPSVFIFPPKPKDILRITRTPEVTCVVLDLGREDPEVQISWFVDGKEVHTAKT
QPREQQFNSTYRVVSVLPIEHQDWLTGKEFKCRVNHIGLPSPIERTISKARGQAHQPGVYVLPPSPKE
LSSSDTVTLTCLIKDFFPPEIDVEWQSNGQPEPESKYHTTAPQLDEDGSYFLYSKLSVDKSRWQQGD
PFTCAVMHEALQNHYTDLSLSHSPGK
CAT_IGG1V1 constant region
SEQ ID NO. 24
TDHPPGPKPCDCPKCPPPEMLGGPSIFIFPPKPKDTLSISRTPEVTCLVVDLGPDDSDVQITWFVDNT
QVYTAKTSPREEQFNSTYRVVSVLPILHQDWLKGKEFKCKVNSKSLPSPIERTISKAKGQPHEPQVYV
LPPAQEELSRNKVSVTCLIKSFHPPDIAVEWEITGQPEPENNYRTTPPQLDSDGTYFVYSKLSVDRSH
WQRGNTYTCSVSHEALHSHHTQKSLTQSPGK
CAT_IGG1V2 constant region
SEQ ID NO. 25
TDHPPGPKPCDCPKCPPPEMLGGPSIFIFPPKPKDTLSISRTPEVTCLVVDLGPDDSDVQITWFVDNT
QVYTAKTSPREEQFNSTYRVVSVLPILHQDWLKGKEFKCKVNSKSLPSPIERTISKAKGQPHEPQVYV
LPPAQEELSRNKVSVTCLIKSFHPPDIAVEWEITGQPEPENNYRTTPPQLDSDGTYFVYSKLSVDRSH
WQRGNTYTCSVSHEALHSHHTQKSLTQSPGK
CAT_IGG2 constant region
SEQ ID NO. 26
KTASTIESKTGEGPKCPVPEIPGAPSVFIFPPKPKDTLSISRTPEVTCLVVDLGPDDSNVQITWFVDNTE
MHTAKTRPREEQFNSTYRVVSVLPILHQDWLKGKEFKCKVNSKSLPSAMERTISKAKGQPHEPQVYV
LPPTQEELSENKVSVTCLIKGFHPPDIAVEWEITGQPEPENNYQTTPPQLDSDGTYFLYSRLSVDRSH
WQRGNTYTCSVSHEALHSHHTQKSLTQSPGK
HORSE_IGHG1 constant region
SEQ ID NO. 27
VIKECNGGCPAECLQVGPSVFIFPPKPKDVLMISRTPTVTCVVVDVGHDFPDVQFNWYVDGVETHTA
TTEPKQEQFNSTYRVVSVLPIQHKDWLSGKEFKCKVNNKALPAPVERTISKPTGQPREPQVYVLAPH
RDELSKNKVSVTCLVKDFYPTDIDIEWKSNGQPEPETKYSTTPAQLDSDGSYFLYSKLTVETNRWQQ
GTTFTCAVMHEALHNHYTEKSVSKSPGK
HORSE_IGHG2 constant region
SEQ ID NO. 28
CVLSAEGVIPIPSVPKPQCPPYTHSKFLGGPSVFIFPPNPKDALMISRTPVVTCVVVNLSDQYPDVQFS
WYVDNTEVHSAITKQREAQFNSTYRVVSVLPIQHQDWLSGKEFKCSVTNVGVPQPISRAISRGKGPS
RVPQVYVLPPHPDELAKSKVSVTCLVKDFYPPDISVEWQSNRWPELEGKYSTTPAQLDGDGSYFLYS
KLSLETSRWQQVESFTCAVMHEALHNHFTKTDISESLGK
HORSE_IGHG3
SEQ ID NO. 29
TTPPCPCECPKCPAPELLGGPSVFIFPPKPKDVLMITRTPEVTCLVVDVSHDSSDVLFTWYVDGTEVK
TAKTMPNEEQNNSTYRVVSVLRIQHQDWLNGKKFKCKVNNQALPAPVERTISKATGQTRVPQVYVLA
PHPDELSKNKVSVTCLVKDFLPTDITVEWQSNEHPEPEGKYRTTEAQKDSDGSYFLYSKLTVETDRW
QQGTTFTCVVMHEALHNHVMQKNVSHSPGK
HORSE_IGHG4 constant region
SEQ ID NO. 30
VIKECNGGCPAECLQVGPSVFIFPPKPKDVLMISRTPTVTCVVVDVGHDFPDVQFNWYVDGVETHTA
TTEPKQEQFNSTYRVVSVLPIQHKDWLSGKEFKCKVNNKALPAPVERTISKPTGQPREPQVYVLAPH
RDELSKNKVSVTCLVKDFYPTDIDIEWKSNGQPEPETKYSTTPAQLDSDGSYFLYSKLTVETNRWQQ
GTTFTCAVMHEALHNHYTEKSVSKSPGK
HORSE_IGHG5 constant region
SEQ ID NO. 31
VVKGSPCPKCPAPELPGGPSVFIFPPKPKDVLKISRKPEVTCVVVDLGHDDPDVQFTWFVDGVETHT
ATTEPKEEQFNSTYRVVSVLPIQHQDWLSGKEFKCSVTNKALPAPVERTTSKAKGQLRVPQVYVLAP
HPDELAKNTVSVTCLVKDFYPPEIDVEWQSNEHPEPEGKYSTTPAQLNSDGSYFLYSKLSVETSRWK
QGESFTCGVMHEAVENHYTQKNVSHSPGK
>HORSE_IGHG6 constant region
SEQ ID NO. 32
KEPCCCPKCPGRPSVFIFPPNPKDTLMISRTPEVTCVVVDVSQENPDVKFNWYVDGVEAHTATTKAK
EKQDNSTYRVVSVLPIQHQDWRRGKEFKCKVNNRALPAPVERTITKAKGELQDPKVYILAPHREEVTK
NTVSVTCLVKDFYPPDINVEWQSNEEPEPEVKYSTTPAQLDGDGSYFLYSKLTVETDRWEQGESFTC
VVMHEAIRHTYRQKSITNFPGK
HORSE_IGHG7 constant region
SEQ ID NO. 33
VIKECGGCPTCPECLSVGPSVFIFPPKPKDVLMISRTPTVTCVVVDVGHDFPDVQFNWYVDGVETHTA
TTEPKQEQNNSTYRVVSILAIQHKDWLSGKEFKCKVNNQALPAPVQKTISKPTGQPREPQVYVLAPHR
DELSKNKVSVTCLVKDFYPTDIDIEWKSNGQPEPETKYSTTPAQLDSDGSYFLYSKLTVETNRWQQG
TTFTCAVMHEALHNHYTEKSVSKSPGK
portion of canine ECD as used in the fusion constructs (without stalk
and without alpha and gamma secretase cleavage 3′ of the stalk region)
SEQ ID NO. 34
KEACPTGLYTHSGECCKACNLGEGVAQPCGANQTVCEPCLDSVTFSDVVSATEPCKPCTE
CVGLQSMSAPCVEADDAVCRCAYGYYQDETTGRCEACRVCEAGSGLVFSCQDRQNTVCEE
CPDGTYSDEANHVDPCLPCTVCEDTERQLRECTRWADAECEEIPKEACPTGLYTHSGECCKACNLG
EGVAQPCGANQTVCEPCLDSVTFSDVVSATEPCKPCTECVGLQSMSAPCVEADDAVCRCAYGYYQ
DETTGRCEACRVCEAGSGLVFSCQDRQNTVCEECPDGTYSDEANHVDPCLPCTVCEDTERQLRECT
RWADAECEEIP
canine p75NTR ECD nucleic acid sequence as used in the fusion
constructs (without stalk and without alpha and gamma secretase
cleavage 3′ of the stalk region)
SEQ ID NO. 35
AAGGAGGCATGTCCCACTGGCCTGTACACCCACAGCGGCGAGTGCTGCAAAGCCTGCAATCTGG
GTGAGGGGGTGGCCCAGCCTTGCGGAGCCAACCAGACCGTGTGTGAGCCCTGCCTGGACAGCG
TGACCTTCTCGGACGTGGTGAGCGCCACCGAGCCGTGCAAGCCGTGCACCGAGTGCGTGGGGC
TGCAGAGCATGTCGGCGCCGTGCGTGGAGGCGGACGACGCCGTGTGCCGCTGCGCCTACGGC
TACTACCAGGACGAGACGACGGGCCGCTGCGAGGCGTGCCGCGTGTGCGAGGCGGGCTCGGG
GCTCGTGTTCTCGTGCCAGGACAGGCAGAACACCGTGTGCGAGGAGTGTCCCGACGGCACGTA
CTCCGACGAGGCCAACCACGTGGACCCGTGCCTGCCCTGCACCGTGTGCGAGGACACCGAGCG
CCAGCTGCGCGAGTGCACGCGCTGGGCCGACGCCGAGTGCGAGGAGATCCCTGGCCGT
bovine p75 NTR protein
SEQ ID NO. 36
KEACLTGLYTHSGECCKACNLGEGVAQPCGANQTVCEPCLDSVTFSDVVSATEPCKPCTECVGLQS
MSAPCVEADDAVCRCAYGYYQDETTGRCEACRVCEAGSGLVFSCQDKQNTVCEECPDGTYSDEAN
HVDPCLPCTVCEDTERQLRECTRWADAECEEIPGRWITRATPPEGSDSTDPSTQEPEVPPEQDLVTS
TVSDVVTTVMGSSQPVVTRGTADNLIPVYCSILAAVVVGLVAYIAFKRWNSCKQNKQGANSRPVNQT
PPPEGEKLHSDSGISVDSQSLHDQQPHTQTAAGQALKGDGGLYSSLPLAKREEVEKLLNGSAGDTW
RHLAGELGYQPEHIDSFTHEACPARALLASWAAQDSATLDTLLAALRRIQRADLVESLCSESTATSPV
ECD is underlined
bovine p75 NTR nucleic acid
SEQ ID NO. 37
ATGGGGTCAGGTGCCGCCGGCCGCGCCATGGACGGGCCGCGCCTGCTGCTGCTGCTGCTGCT
GCTCCTGGGGGTGTCCCTTGGAGGTGCCAAGGAAGCATGCCTCACGGGCCTGTACACCCACAG
CGGAGAGTGCTGCAAAGCCTGCAACCTGGGCGAGGGTGTGGCCCAGCCTTGTGGAGCCAACCA
GACCGTGTGTGAACCCTGCCTGGACAGCGTGACCTTCTCGGACGTGGTGAGCGCCACGGAGCC
GTGTAAGCCGTGCACGGAGTGCGTGGGACTGCAGAGCATGTCGGCGCCCTGCGTGGAGGCCGA
CGACGCCGTGTGCCGCTGCGCCTACGGCTATTACCAGGACGAGACGACCGGCCGCTGCGAGGC
GTGCCGCGTGTGCGAGGCGGGCTCGGGGCTCGTGTTCTCGTGCCAGGACAAGCAGAACACCGT
CTGCGAGGAGTGCCCCGACGGCACGTACTCCGACGAGGCCAACCACGTGGACCCCTGCCTGCC
CTGCACGGTGTGCGAGGACACGGAGCGCCAGCTGCGCGAGTGCACGCGCTGGGCCGACGCCG
AGTGCGAGGAGATCCCTGGACGTTGGATTACACGGGCCACGCCCCCTGAGGGCTCCGACAGCA
CAGACCCCAGCACCCAGGAGCCCGAGGTACCTCCAGAGCAAGATCTGGTAACCAGCACTGTGTC
AGATGTGGTGACCACGGTGATGGGCAGCTCCCAGCCTGTGGTGACCCGAGGTACCGCCGACAA
CCTCATCCCTGTCTATTGCTCCATCCTGGCTGCTGTGGTTGTGGGCCTTGTGGCCTACATCGCCT
TCAAGAGGTGGAACAGCTGCAAGCAGAACAAGCAAGGAGCCAACAGCCGACCTGTGAACCAGAC
ACCCCCACCAGAGGGGGAAAAGCTACACAGCGATAGCGGCATCTCTGTGGACAGCCAGAGCCT
GCATGACCAGCAGCCCCACACGCAGACTGCCGCAGGCCAGGCCCTCAAGGGTGATGGAGGCCT
CTACAGCAGCCTGCCGCTGGCCAAGCGGGAGGAGGTGGAGAAGCTGCTCAACGGCTCTGCGGG
GGACACCTGGCGGCATCTGGCAGGCGAGTTGGGTTACCAGCCTGAGCACATAGACTCCTTCACC
CACGAGGCCTGCCCAGCCCGCGCCCTGCTGGCCAGCTGGGCTGCCCAGGACAGCGCCACGCT
CGACACCCTCCTTGCGGCCCTGCGCCGCATCCAGCGCGCCGACATCGTGGAGAGCCTGTGCAG
CGAGTCCACGGCCACGTCCCCCGTGTGA
feline p75NTR protein ECD
SEQ ID NO. 38
KEACPTGLFTHSGECCKACNLGEGVAQPCGANQTVCEPCLDSVTFSDVVSATEPCKPCTE
CVGLQSMSAPCVEADDAVCRCAYGYYQDETTGRCEACRVCEAGSGLVFSCQDRQNTVCEE
CPDGTYSDEANHVDPCLPCTVCEDTERQLRECTRWADAECEEIPGRWITRSTPSEGSDST
APSTEEPEVPPEQDLIASTVADVVTTVMGSSQPVVTRGTADN
feline p75NTR ECD-feline IgG1 wt Fc protein fusion
SEQ ID NO. 39
MDGPRPLLLLLPLLLGVSLGGAKEACPTGLFTHSGECCKACNLGEGVAQPCGANQTVCEPCLDSVTF
SDVVSATEPCKPCTECVGLQSMSAPCVEADDAVCRCAYGYYQDETTGRCEACRVCEAGSGLVFSC
QDRQNTVCEECPDGTYSDEANHVDPCLPCTVCEDTERQLRECTRWADAECEEIPGRGGGGVPKTA
STIESKTCDCPKCPVPEIPGAPSVFIFPPKPKDTLSISRTPEVTCLVVDLGPDDSNVQITWFVDNTEMHT
AKTRPREEQFNSTYRVVSVLPILHQDWLKGKEFKCKVNSKSLPSAMERTISKAKGQPHEPQVYVLPPT
QEELSENKVSVTCLIKGFHPPDIAVEWEITGQPEPENNYQTTPPQLDSDGTYFLYSRLSVDRSHWQR
GNTYTCSVSHEALHSHHTQKSLTQSPGK
Signal peptide
Feline p75-ECD
Linker GGGG
Feline Fc-IgG1
feline p75NTR ECD-feline IgG1 wt Fc nucleic acid sequence
SEQ ID NO. 40
ATGGATGGACCTAGACCTCTGCTGCTGCTCCTGCCTCTGCTGTTGGGAGTTTCTCTCGGCGGAG
CCAAAGAGGCTTGTCCTACCGGCCTGTTTACCCACTCTGGCGAGTGTTGCAAGGCCTGTAATCTC
GGCGAAGGCGTGGCACAACCTTGTGGCGCTAATCAGACAGTGTGCGAGCCTTGCCTGGACTCC
GTGACCTTCTCTGATGTGGTGTCTGCCACCGAGCCATGCAAGCCTTGTACCGAGTGTGTGGGCC
TGCAGTCCATGTCTGCCCCTTGTGTGGAAGCCGACGACGCCGTGTGTAGATGTGCCTACGGCTA
CTACCAGGACGAGACAACCGGAAGATGCGAGGCCTGCAGAGTGTGTGAAGCTGGCTCTGGACT
GGTGTTCTCCTGCCAAGACAGACAGAACACCGTGTGCGAGGAATGCCCTGACGGCACCTACTCT
GATGAGGCCAATCACGTGGACCCCTGCCTGCCTTGTACTGTGTGCGAAGATACCGAGCGGCAGC
TGCGCGAGTGTACCAGATGGGCTGATGCCGAGTGCGAAGAGATCCCTGGAAGAGGCGGAGGCG
GAGTGCCTAAGACCGCTTCTACCATCGAGTCCAAGACCTGCGACTGCCCTAAGTGCCCTGTGCC
TGAAATTCCTGGCGCTCCCTCCGTGTTCATCTTCCCACCTAAGCCTAAGGACACCCTGTCCATCT
CTCGGACCCCTGAAGTGACCTGCCTGGTGGTTGATCTGGGCCCTGACGACTCCAACGTGCAGAT
CACTTGGTTTGTGGACAACACCGAGATGCACACCGCCAAGACCAGACCTAGAGAGGAACAGTTC
AACTCCACCTACAGAGTGGTGTCCGTGCTGCCCATCCTGCACCAGGATTGGCTGAAGGGCAAAG
AATTCAAGTGCAAAGTGAACTCCAAGAGCCTGCCTTCCGCCATGGAACGGACCATCTCTAAGGCT
AAGGGCCAGCCTCATGAGCCCCAGGTGTACGTTCTGCCTCCTACACAAGAGGAACTGTCCGAGA
ACAAAGTGTCCGTGACATGCCTGATCAAGGGCTTTCACCCTCCTGATATCGCCGTGGAATGGGA
GATCACCGGACAGCCTGAGCCTGAGAACAACTACCAGACCACACCTCCTCAGCTGGACAGCGAC
GGAACCTACTTCCTGTACTCCCGGCTGTCCGTGGACAGATCCCATTGGCAGAGAGGCAACACCT
ACACCTGTTCCGTGTCTCACGAGGCCCTGCACTCTCATCACACCCAGAAGTCCCTGACACAGTCC
CCTGGCAAG
feline IgG3
SEQ ID NO: 41
LPPCKCPKCPVPEIPGGPSVFIFPPKPKDTLSISRTPEVTCLVVDLGPDDSNVQITWFVDNTEMHTAKT
RPREEQFNSTYRVVSVLPIVHQDWLTGKEFKCKVNSKALPSAIERTISKAKGQPHEPQVYVLPPAQEE
LSENKVCVTCLIKGFYPPDIAVEWEITGQPEPENNYRTTPPQLDSDGTYFVYSRLSMDRSRWQSGNT
YTCSVSHEALHSHHTQKSLTQSPGK

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An isolated companion animal p75NTR protein or a portion thereof.

2. The isolated companion animal p75NTR protein or a portion thereof according to claim 1 wherein the companion animal is a cat, dog, pig, cow, or horse.

3. The isolated companion animal p75NTR protein or a portion thereof according to claim 2 wherein the companion animal is a dog.

4. The isolated companion animal p75NTR protein or a portion thereof according to claim 2 wherein the companion animal is a cat.

5. The isolated companion animal p75NTR protein or a portion thereof according to claim 3 comprising SEQ ID NO. 1.

6. The isolated companion animal p75NTR protein or a portion thereof according to claim 4 comprising SEQ ID NO. 3.

7. The isolated companion animal p75NTR protein or a portion thereof according to claim 1 wherein the p75NTR protein or a portion thereof comprises the extracellular domain or part thereof.

8. The isolated companion animal p75NTR extracellular domain according to claim 7 wherein the companion animal is a dog and the p75NTR extracellular domain comprises SEQ ID NO. 7.

9. The isolated companion animal p75NTR extracellular domain according to claim 7 wherein the companion animal is a cat and the p75NTR extracellular domain comprises SEQ ID NO. 38.

10. An isolated nucleic acid encoding a companion animal p75NTR protein or a portion thereof according to claim 1.

11. The isolated nucleic acid according to claim 10 wherein the companion animal is a dog, and the nucleic acid comprises SEQ ID NO. 2.

12. The isolated nucleic acid according to claim 10 wherein the companion animal is a cat, and the nucleic acid comprises SEQ ID NO. 4.

13. A vector comprising a nucleic acid according to any one of claims 10 through 12.

14. A host cell comprising a nucleic acid according to any one of claims 10 through 12 or a vector according to claim 13.

15. A fusion protein comprising an isolated companion animal p75NTR extracellular domain or portion thereof and a half-life extending moiety.

16. The fusion protein according to claim 15 wherein the half-life extending moiety is selected from an Fc domain, a serum albumin binder, or PEG.

17. The fusion protein according to claim 16 wherein the half-life extending moiety is a wild type or mutant Fc domain.

18. The fusion protein according to claim(s) 15 wherein the half-life extending moiety is an Fc domain and the p75NTR extracellular domain or portion thereof and the Fc domain are linked with a linker.

19. The fusion protein according to claim 18 wherein the linker is a peptide linker.

20. The fusion protein according to claim 19 wherein the peptide linker is (G₄S)ₙ wherein n is 1 to 4.

21. The fusion protein according to claim 15 wherein the companion animal is a cat, dog or horse.

22. The fusion protein according to claim 21 wherein the companion animal is a dog and the p75NTR extracellular domain comprises SEQ ID No. 7 or a portion thereof such as SEQ ID No. 34.

23. (canceled)

24. The fusion protein according to claim 22 wherein the Fc domain is a canine Fc domain.

25. The fusion protein according to claim 24 comprising SEQ ID NO. 11 or SEQ ID NO.13.

26. The fusion protein according to claim 21 wherein the companion animal is a cat and the p75NTR extracellular domain comprises SEQ ID NO. 38.

27. (canceled)

28. The fusion protein according to claim 26 comprising SEQ ID NO. 39.

29. A nucleic acid encoding a fusion protein according to claim 15.

30. A vector comprising a nucleic acid according to claim 29.

31. A host cell comprising a nucleic acid according to claim 29 or a vector according to claim 30.

32. A pharmaceutical composition comprising an isolated companion animal p75NTR protein according to claim 1, or a fusion protein according to claim 15.

33. A method for treating pain in a companion animal comprising administering the pharmaceutical composition according to claim 32.

34. (canceled)

35. (canceled)

36. (canceled)

37. (canceled)

38. A method of inhibiting NGF activity in a companion animal comprising administering to a companion animal the pharmaceutical composition of claim 32.

39. The method of claim 38 comprising administration of a second compound that treats pain.

40. A kit comprising the pharmaceutical composition of claim 32 and optionally instructions for use.


SEQUENCE LISTING (41 sequences)

SEQ ID NO: 1 - protein, 399 aa, Canis sp.
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEDSDSTAPSTEEPELPPDQEIIASTMADVVTTVMGSSQPVVTRGTADNLIPVYCSILAAVVVGLVAYIAFKRWNSCKQNKQGANSRPVNQTPPPEGEKLHSDSGISVDSQSLHDQQPHTQTAAGQALKGDGGLYSSLPPAKREEVEKLLNGSAGDTWRHLAGELGYQPEHIDSFTHEACPARALLASWAAQDSATLDALLAALRRIQRADIVESLCSESTATSPV

SEQ ID NO: 2 - DNA, 1263 bp, Canis sp.
  source 1..1263: mol_type = genomic DNA
atggacgggccgcgcctgctgctgctgctgctgctgctcctgggggtgtcccttggaggtgccaaggaggcatgtcccactggcctgtacacccacagcggcgagtgctgcaaagcctgcaatctgggtgagggggtggcccagccttgcggagccaaccagaccgtgtgtgagccctgcctggacagcgtgaccttctcggacgtggtgagcgccaccgagccgtgcaagccgtgcaccgagtgcgtggggctgcagagcatgtcggcgccgtgcgtggaggcggacgacgccgtgtgccgctgcgcctacggctactaccaggacgagacgacgggccgctgcgaggcgtgccgcgtgtgcgaggcgggctcggggctcgtgttctcgtgccaggacaggcagaacaccgtgtgcgaggagtgtcccgacggcacgtactccgacgaggccaaccacgtggacccgtgcctgccctgcaccgtgtgcgaggacaccgagcgccagctgcgcgagtgcacgcgctgggccgacgccgagtgcgaggagatccctggccgttggattacccggtccacaccctcagaggactcggacagcaccgcccccagcacagaggagccagagctacctccagatcaagaaatcatagccagcaccatggcagatgtggtgaccacagtgatgggcagctctcagcctgtagtgacccgaggaaccgctgacaacctcatccctgtctactgctccatcctggccgccgtggttgtgggcttagtggcctacattgccttcaagaggtggaacagctgcaagcagaacaagcaaggagccaacagccggcccgtgaaccagacgcctccgccggagggagaaaagctccacagtgacagtggcatctctgtggacagccagagcctgcatgaccagcagccccacacacagacggccgcaggccaggccctcaagggggatggaggtctctacagcagcctgccaccagccaagcgggaggaggtggagaagctgctcaatggctctgcgggggacacctggcggcacctggcaggtgagctgggctaccagcctgagcacatagactccttcacccacgaggcctgcccagcccgagccctgcttgccagctgggccgcccaggacagcgcgacgctcgacgccctcctggctgctctgcgccgcatccagcgagccgacatcgtggagagcctgtgtagcgagtccacggccacgtctccagtgtga

SEQ ID NO: 3 - protein, 399 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEGSDSTAPSTEEPEVPPEQDLIASTVADVVTTVMGSSQPVVTRGTADNLIPVYCSILAAVVVGLVAYIAFKRWNSCKQDKQGANSRPVNQTPPPEGEKLHSDSGISVDSQSLHDQQSHTQTAAGQALKGDGGLYSSLPSAKREEVEKLLNGSAGDTWRHLAGELGYQPEHIDSFTREACPARALLASWAAQDSATLDALLAALRRIQRADIVESLCSESTATSPV

SEQ ID NO: 4 - DNA, 1266 bp, Felis catus
  source 1..1266: mol_type = genomic DNA
atggacgggccgcgcccgctgctgctgctgttgccgctgctcctgggggtgtcccttggaggtgccaaggaggcatgtcccacgggcctgttcacccacagcggcgagtgctgtaaagcctgcaacctgggagagggcgtagcccagccttgcggagccaaccagaccgtgtgtgagccctgcctggacagcgtgaccttctcggacgtggtgagcgccacggagccgtgcaagccgtgcaccgagtgcgtgggcctgcagagcatgtcggcgccgtgcgtggaggccgacgacgccgtgtgtcgctgcgcctacggctactaccaggacgagacgacgggccgctgcgaggcgtgccgcgtgtgcgaggcgggctccggcctggtgttctcgtgccaggaccggcagaataccgtgtgcgaggagtgtcccgacggcacgtactcggacgaggccaaccacgtggacccgtgcctgccctgcaccgtgtgcgaggacaccgagcgccagctgcgcgagtgcacgcgctgggccgacgccgagtgcgaggagatccctggccgttggattactcggtctacaccttcggagggctcggacagcaccgcccccagcacggaggagccagaggtacctccagagcaagacctcatagccagcacggtggcagatgtggtgaccacagtgatgggcagctctcagcccgtagtgacccgaggcaccgccgacaacctcatccctgtctattgttccatcctggccgctgtggttgtgggcctggtggcctacattgccttcaagaggtggaacagctgcaaacaggacaagcaaggcgccaacagccggcccgtgaaccagacgcccccgcccgagggagaaaagctccacagtgacagtggcatctctgtggacagccagagcctgcatgaccagcagtcccacacgcagacggccgccggccaggccctcaagggggacggaggtctctacagcagcctgccgtcagccaagcgggaggaggtagagaaactgctcaacggctctgcgggggacacgtggcggcacctggcgggcgagctgggctaccagcctgagcacatagactccttcacccgcgaggcctgcccagcccgggccctgctcgccagctgggccgcccaggacagcgcgacgctcgacgccctcctggccgccctgcgccgcatccagcgggccgacatcgtcgagagcctgtgcagcgagtccacggccacgtccccggtgtga

SEQ ID NO: 5 - protein, 405 aa, Equus caballus
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFSCQDKQNTVCEECPDGTYSDEANHVDPCLPCTVCEDTERQLRECTRWADAECEEIPSRWITRATPPEGSDSTAPSTQEPEGPPEKDLVASTVADVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTAAGQALKGDGGLYSSLPLAKREEVEKLLNGSAGDTWRHLAGLVGQGLLRLELVSVFQGPAHGGMLPPATPSLQAPVWLGPEGCSEKWDQRGNAARRAGLRVWPMEGLSQV

SEQ ID NO: 6 - DNA, 1290 bp, Equus caballus
  source 1..1290: mol_type = genomic DNA
atgagggcaggtgccgccgactgcgccatggacggaccgcgccttctgctgctgcttctgctcttgggggtgtgcctgctgggaggtgccaaggaggtgtgccccacagacctgtacacccacagcggcgagtgctgcaaagcctgcaacctgggcgagggtgtggcccagccttgcggagccaaccagactgtgtgtgaaccctgcctggacagcgtgacgttctcggacgtggtgagcgccacagagccatgtaagccgtgcaccgagtgcgtgggcctgcagagcatgtcggcgccatgcgtggaggccgacgacgcggtgtgccgctgcgcctatggctactaccaggacgagacgacgggccgctgcgaggcgtgccaggtgtgcgaggcgggctcgggcctcgtgttctcgtgccaggacaagcagaacaccgtgtgcgaggaatgccccgacggcacgtactccgacgaggccaaccacgtggacccgtgcctgccctgcaccgtgtgcgaggacaccgagcgccagctgcgagagtgcacgcgctgggccgacgccgagtgcgaggagatccccagccgttggattacacgggccacgccgccggagggctcagacagcactgcccccagcacccaggagcccgagggacctccagagaaagaccttgtagccagcacggtggcggatgtggtgaccacagtgatgggcagctctcagcccgtggtgacccgaggcaccacggacaacctcatccccgtctattgctccatcctggccgctgtggttgtgggccttgtggcctacatcgccttcaagaggtggaacagctgcaagcagaacaagcaaggagccaacagccgacccgtgaaccagacaccaccacccgagggagaaaaactccacagcgacagcggcatctctgtggacagccagagcctgcatgaccagcagcctcacacacagacagccgcaggccaggccctcaagggagatggaggcctctacagcagcctgccactggccaagagggaagaggtggagaagctactcaatggctccgcaggggacacctggcggcacctggcgggtgagctgggctaccagcccgagcacatagactccttcacccacgaggcctgccccgtccgcgccctgcttgccagctgggccgcccaggacagtgcgacattcgatgccctcctgaccgccctgcgccgcatccagcgagccgacattgtcgagagcctgtgcagcgagtccaccgccacatccccggtgtga

SEQ ID NO: 7 - protein, 222 aa, Canis sp.
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEDSDSTAPSTEEPELPPDQEIIASTMADVVTTVMGSSQPVVTRGTADN

SEQ ID NO: 8 - DNA, 666 bp, Canis sp.
  source 1..666: mol_type = genomic DNA
aaggaggcatgtcccactggcctgtacacccacagcggcgagtgctgcaaagcctgcaatctgggtgagggggtggcccagccttgcggagccaaccagaccgtgtgtgagccctgcctggacagcgtgaccttctcggacgtggtgagcgccaccgagccgtgcaagccgtgcaccgagtgcgtggggctgcagagcatgtcggcgccgtgcgtggaggcggacgacgccgtgtgccgctgcgcctacggctactaccaggacgagacgacgggccgctgcgaggcgtgccgcgtgtgcgaggcgggctcggggctcgtgttctcgtgccaggacaggcagaacaccgtgtgcgaggagtgtcccgacggcacgtactccgacgaggccaaccacgtggacccgtgcctgccctgcaccgtgtgcgaggacaccgagcgccagctgcgcgagtgcacgcgctgggccgacgccgagtgcgaggagatccctggccgttggattacccggtccacaccctcagaggactcggacagcaccgcccccagcacagaggagccagagctacctccagatcaagaaatcatagccagcaccatggcagatgtggtgaccacagtgatgggcagctctcagcctgtagtgacccgaggaaccgctgacaac

SEQ ID NO: 9 - protein, 42 aa, Canis sp.
WITRSTPSEDSDSTAPSTEEPELPPDQEIIASTMADVVTTVM

SEQ ID NO: 10 - DNA, 126 bp, Canis sp.
  source 1..126: mol_type = genomic DNA
tggattacccggtccacaccctcagaggactcggacagcaccgcccccagcacagaggagccagagctacctccagatcaagaaatcatagccagcaccatggcagatgtggtgaccacagtgatg

SEQ ID NO: 11 - protein, 422 aa, Canis sp.
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 12 - DNA, 1269 bp, Canis sp.
  source 1..1269: mol_type = genomic DNA
atggaatggtcctgggtgttcctgttcttcctgtccgtgaccaccggcgtgcactccaaagaggcttgtcctaccggcctgtacacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagatccctggaggtggcggacgcgagaatggcagagtgcctagacctcctgactgccctaagtgccctgctcctgaaatgctcggcggaccctccgtgttcatcttcccacctaagcctaaggacaccctgctgatcgctcggacccctgaagtgacatgcgtggtggtggatctggaccccgaggatcctgaggtgcagatcagttggttcgtggacggcaagcagatgcagaccgctaagacccagcctagagaggaacagttcaacggcacctacagagtggtgtctgtgctgcctatcggccaccaggattggctgaagggcaagcagtttacctgcaaagtgaacaacaaggccctgccttctccaatcgagcggaccatctctaaggccagaggccaggctcatcagccttccgtgtatgtcctgccacctagccgcgaggaactgtccaagaacaccgtgtctctgacctgcctgatcaaggacttcttccctcctgacatcgacgtggaatggcagtccaacggccagcaagagcccgagtctaagtaccggacaacccctccacagctggacgaggacggctcctacttcctgtactccaagctgtccgtggacaagtctcggtggcagagaggcgacaccttcatctgtgctgtgatgcacgaggccctgcacaaccactacacccagaagtccctgtctcactcccctggcaagtga

SEQ ID NO: 13 - protein, 422 aa, Canis sp.
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGGGGRENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 14 - DNA, 1269 bp, Canis sp.
  source 1..1269: mol_type = genomic DNA
atggaatggtcctgggtgttcctgttcttcctgtccgtgaccaccggcgtgcactccaaagaggcttgtcctaccggcctgtacacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagatccctggaggtggcggacgcgagaatggcagagtgcctagacctcctgactgccctaagtgccctgctcctgaaatgctcggcggaccctccgtgttcatcttcccacctaagcctaaggacaccctgtatatcactcgggaacctgaagtgacatgcgtggtggtggatctggaccccgaggatcctgaggtgcagatcagttggttcgtggacggcaagcagatgcagaccgctaagacccagcctagagaggaacagttcaacggcacctacagagtggtgtctgtgctgcctatcggccaccaggattggctgaagggcaagcagtttacctgcaaagtgaacaacaaggccctgccttctccaatcgagcggaccatctctaaggccagaggccaggctcatcagccttccgtgtatgtcctgccacctagccgcgaggaactgtccaagaacaccgtgtctctgacctgcctgatcaaggacttcttccctcctgacatcgacgtggaatggcagtccaacggccagcaagagcccgagtctaagtaccggacaacccctccacagctggacgaggacggctcctacttcctgtactccaagctgtccgtggacaagtctcggtggcagagaggcgacaccttcatctgtgctgtgatgcacgaggccctgcacaaccactacacccagaagtccctgtctcactcccctggcaagtga

SEQ ID NO: 15 - protein, 471 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 16 - protein, 475 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 17 - protein, 473 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSQSGSTVALACLVSGYIPEPVTVSWNSGSLTSGVHTFPSILQSSGLYSLSSMVTVPSSRWPSETFTCNVAHPATNTKVDKPVVKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 18 - protein, 471 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASSTAPSVFPLAPSCGSTSGSTVALACLVSGYFPEPVTVSWNSGSLTSGVHTFPSVLKSSGLYSLSSMVTVPSSRLPSETFTCNVVHPATNTKVDKPVPKESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 19 - protein, 233 aa, Canis sp.
FNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 20 - protein, 235 aa, Canis sp.
RENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 21 - protein, 235 aa, Canis sp.
RENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 22 - protein, 233 aa, Canis sp.
ECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 23 - protein, 231 aa, Canis sp.
ESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 24 - protein, 235 aa, Felis catus
TDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 25 - protein, 235 aa, Felis catus; identical to SEQ ID NO: 24

SEQ ID NO: 26 - protein, 236 aa, Felis catus
KTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 27 - protein, 229 aa, Equus caballus
VIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 28 - protein, 242 aa, Equus caballus
CVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 29 - protein, 232 aa, Equus caballus
TTPPCPCECPKCPAPELLGGPSVFIFPPKPKDVLMITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 30 - protein, 229 aa, Equus caballus; identical to SEQ ID NO: 27

SEQ ID NO: 31 - protein, 230 aa, Equus caballus
VVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 32 - protein, 224 aa, Equus caballus
KEPCCCPKCPGRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 33 - protein, 230 aa, Equus caballus
VIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 34 - protein, 328 aa, Canis sp.
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIP

SEQ ID NO: 35 - DNA, 498 bp, Canis sp.
  source 1..498: mol_type = genomic DNA
aaggaggcatgtcccactggcctgtacacccacagcggcgagtgctgcaaagcctgcaatctgggtgagggggtggcccagccttgcggagccaaccagaccgtgtgtgagccctgcctggacagcgtgaccttctcggacgtggtgagcgccaccgagccgtgcaagccgtgcaccgagtgcgtggggctgcagagcatgtcggcgccgtgcgtggaggcggacgacgccgtgtgccgctgcgcctacggctactaccaggacgagacgacgggccgctgcgaggcgtgccgcgtgtgcgaggcgggctcggggctcgtgttctcgtgccaggacaggcagaacaccgtgtgcgaggagtgtcccgacggcacgtactccgacgaggccaaccacgtggacccgtgcctgccctgcaccgtgtgcgaggacaccgagcgccagctgcgcgagtgcacgcgctgggccgacgccgagtgcgaggagatccctggccgt

SEQ ID NO: 36 - protein, 399 aa, Bos taurus
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRATPPEGSDSTDPSTQEPEVPPEQDLVTSTVSDVVTTVMGSSQPVVTRGTADNLIPVYCSILAAVVVGLVAYIAFKRWNSCKQNKQGANSRPVNQTPPPEGEKLHSDSGISVDSQSLHDQQPHTQTAAGQALKGDGGLYSSLPLAKREEVEKLLNGSAGDTWRHLAGELGYQPEHIDSFTHEACPARALLASWAAQDSATLDTLLAALRRIQRADLVESLCSESTATSPV

SEQ ID NO: 37 - DNA, 1290 bp, Bos taurus
  source 1..1290: mol_type = genomic DNA
atggggtcaggtgccgccggccgcgccatggacgggccgcgcctgctgctgctgctgctgctgctcctgggggtgtcccttggaggtgccaaggaagcatgcctcacgggcctgtacacccacagcggagagtgctgcaaagcctgcaacctgggcgagggtgtggcccagccttgtggagccaaccagaccgtgtgtgaaccctgcctggacagcgtgaccttctcggacgtggtgagcgccacggagccgtgtaagccgtgcacggagtgcgtgggactgcagagcatgtcggcgccctgcgtggaggccgacgacgccgtgtgccgctgcgcctacggctattaccaggacgagacgaccggccgctgcgaggcgtgccgcgtgtgcgaggcgggctcggggctcgtgttctcgtgccaggacaagcagaacaccgtctgcgaggagtgccccgacggcacgtactccgacgaggccaaccacgtggacccctgcctgccctgcacggtgtgcgaggacacggagcgccagctgcgcgagtgcacgcgctgggccgacgccgagtgcgaggagatccctggacgttggattacacgggccacgccccctgagggctccgacagcacagaccccagcacccaggagcccgaggtacctccagagcaagatctggtaaccagcactgtgtcagatgtggtgaccacggtgatgggcagctcccagcctgtggtgacccgaggtaccgccgacaacctcatccctgtctattgctccatcctggctgctgtggttgtgggccttgtggcctacatcgccttcaagaggtggaacagctgcaagcagaacaagcaaggagccaacagccgacctgtgaaccagacacccccaccagagggggaaaagctacacagcgatagcggcatctctgtggacagccagagcctgcatgaccagcagccccacacgcagactgccgcaggccaggccctcaagggtgatggaggcctctacagcagcctgccgctggccaagcgggaggaggtggagaagctgctcaacggctctgcgggggacacctggcggcatctggcaggcgagttgggttaccagcctgagcacatagactccttcacccacgaggcctgcccagcccgcgccctgctggccagctgggctgcccaggacagcgccacgctcgacaccctccttgcggccctgcgccgcatccagcgcgccgacatcgtggagagcctgtgcagcgagtccacggccacgtcccccgtgtga

SEQ ID NO: 38 - protein, 222 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEGSDSTAPSTEEPEVPPEQDLIASTVADVVTTVMGSSQPVVTRGTADN

SEQ ID NO: 39 - protein, 430 aa, Felis catus
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRGGGGVPKTASTIESKTCDCPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 40 - DNA, 1290 bp, Felis catus
  source 1..1290: mol_type = genomic DNA
atggatggacctagacctctgctgctgctcctgcctctgctgttgggagtttctctcggcggagccaaagaggcttgtcctaccggcctgtttacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagatccctggaagaggcggaggcggagtgcctaagaccgcttctaccatcgagtccaagacctgcgactgccctaagtgccctgtgcctgaaattcctggcgctccctccgtgttcatcttcccacctaagcctaaggacaccctgtccatctctcggacccctgaagtgacctgcctggtggttgatctgggccctgacgactccaacgtgcagatcacttggtttgtggacaacaccgagatgcacaccgccaagaccagacctagagaggaacagttcaactccacctacagagtggtgtccgtgctgcccatcctgcaccaggattggctgaagggcaaagaattcaagtgcaaagtgaactccaagagcctgccttccgccatggaacggaccatctctaaggctaagggccagcctcatgagccccaggtgtacgttctgcctcctacacaagaggaactgtccgagaacaaagtgtccgtgacatgcctgatcaagggctttcaccctcctgatatcgccgtggaatgggagatcaccggacagcctgagcctgagaacaactaccagaccacacctcctcagctggacagcgacggaacctacttcctgtactcccggctgtccgtggacagatcccattggcagagaggcaacacctacacctgttccgtgtctcacgaggccctgcactctcatcacacccagaagtccctgacacagtcccctggcaag

SEQ ID NO: 41 - protein, 229 aa, Felis catus
LPPCKCPKCPVPEIPGGPSVFIFPPKPKDTLSISRTPEVTCLVVDLGPDDSNVQITWFVDNTEMHTAKTRPREEQFNSTYRVVSVLPIVHQDWLTGKEFKCKVNSKALPSAIERTISKAKGQPHEPQVYVLPPAQEELSENKVCVTCLIKGFYPPDIAVEWEITGQPEPENNYRTTPPQLDSDGTYFVYSRLSMDRSRWQSGNTYTCSVSHEALHSHHTQKSLTQSPGK